(19) United States
(12) Patent Application Publication
(10) Pub. No.: US 2026/0110695 A1
(43) Pub. Date: Apr. 23, 2026

(54) Methods for Standardizing Lectin Reagents, IgA1 Calibration Standards, and Quantitative Measurement of Galactose-Deficient IgA1 in Human Samples

(71) Applicant: Reliant Glycosciences, LLC, Birmingham, AL (US)

(72) Inventors: William Placzek, Vestavia Hills, AL (US); Thomasz Szul, Leeds, AL (US); Matthew Renfrow, Birmingham, AL (US); Janusz Tucholski, Vestavia Hills, AL (US); Jan Novak, Pelham, AL (US)

(73) Assignee: Reliant Glycosciences, LLC, Birmingham, AL (US)

(21) Appl. No.: 19/366,152

(22) Filed: Oct. 22, 2025

Related U.S. Application Data
(60) Provisional application No. 63/710,563, filed on Oct. 22, 2024.

(51) Int. Cl. G01N 33/68; C07K 16/28; C12N 9/24; G01N 21/78; G01N 33/543

(52) U.S. Cl. CPC G01N 33/6857; C07K 16/283; C12N 9/2402; G01N 21/78; G01N 33/543; C07K 2317/40; C12Y 302/01018; G01N 2333/4724; G01N 2470/04

(57) ABSTRACT

Methods, systems, and kits are provided for standardizing lectin reagents and measuring galactose-deficient IgA1 (Gd-IgA1) as a biomarker for IgA nephropathy. The invention includes a method for standardizing test batches of lectin reagents by comparing binding activity to reference batches using calibration standards, with acceptance criteria based on analytical recovery within thresholds. A stabilized reference standard comprising enzymatically modified or recombinantly produced Gd-IgA1 exhibits parallelism with natural serum samples and enables reproducible quantification across runs. The invention further provides a lectin-based ELISA assay kit for quantitative measurement of Gd-IgA1 in biological samples, particularly serum, using standardized N-acetylgalactosamine (GalNAc)-specific lectins such as biotinylated Helix pomatia agglutinin (HPA) or Helix aspersa agglutinin (HAA). Clinical applications include diagnosing IgA nephropathy, risk stratification, monitoring disease progression, assessing treatment response, and screening kidney transplant donors. The standardization methodology is broadly applicable to validating binding reagents for detecting diverse biomarkers, peptides, and proteins in diagnostic assays.

### CROSS-REFERENCE TO RELATED APPLICATIONS

This application claims priority to U.S. Provisional Patent Application Ser. No. 63/710,563, filed Oct. 22, 2024, the entirety of which are incorporated by reference as if fully disclosed herein.

### FIELD OF THE INVENTION

The present application relates generally to the detection and quantification of Gal-Deficient IgA1; for example, to identify subjects with IgA nephropathy. In various embodiments, the invention relates to one or more biomarkers, methods, devices, reagents, systems, and kits for characterizing Gal-Deficient IgA1 in a subject. In various embodiments, the invention relates to a method of standardizing reagents, such as lectin, for use in an assay.

### GENERAL BACKGROUND

IgA nephropathy (IgAN), also known as Berger's disease, is the most common form of primary glomerulonephritis worldwide and a major cause of kidney failure. IgA nephropathy is a mesangioproliferative glomerular disease defined by characteristic IgA1 mesangial deposits. Immunoglobulin A (IgA) is an antibody that plays a role in the immune function of mucous membranes.

IgA1 mesangial deposits likely originate from circulating immune complexes that contain IgA1 with Galactose (Gal)-deficient O-glycans (Gd-IgA1, the autoantigen) that are bound by IgG autoantibodies. (Suzuki, H., et al., The pathophysiology of IgA nephropathy. J. Am. Soc. Nephrol., 2011. 22: p. 1795-1803; Suzuki, H., et al., Aberrantly glycosylated IgA1 in IgA nephropathy patients is recognized by IgG antibodies with restricted heterogeneity. J Clin Invest, 2009. 119(6): p. 1668-77). IgA nephropathy frequently manifests with episodes of macroscopic hematuria or acute nephritic syndrome that coincide with mucosal infections. The incidence of IgA nephropathy peaks in young adults and can reduce life expectancy by as much as 10 years.

Diagnosing kidney disease, such as IgA nephropathy, is a complicated process that most often starts with either a standard urinalysis or the occurrence of cloudy (proteinuria) and/or bloody (hematuria) urine. The subsequent evaluation process usually includes measurement of the serum creatinine and determination of the estimated glomerular filtration rate (eGFR) and review of the patient's pertinent medical history and demographics to develop the differential diagnosis decision tree. For most of the possible outcomes, the process culminates in a kidney biopsy. However, kidney biopsies entail significant risk for bleeding complications that may require transfusion, embolization of the bleeding vessel, removal of the bleeding biopsied kidney, and, rarely, death. As a consequence, biopsy is sometimes delayed while determining if the proteinuria or hematuria persists. Also, there is the possibility that the biopsy yields inconclusive results if an insufficient specimen is obtained. Kidney biopsy is the only means to diagnose IgA nephropathy because there is no other disease-specific test for IgA nephropathy. Currently, there is no direct method of monitoring IgA nephropathy without kidney biopsy; Doctors can only monitor the rate of kidney function decline. Thus, there is a clear unmet need for a non-invasive means to diagnose and directly monitor this chronic kidney condition with disease-specific biomarkers. Major issues in the field currently include the lack of non-invasive means of diagnosing the disease, the early identification of patients at risk for disease progression, and development of effective therapy to allow early intervention for such patients. Further, current standard of care for IgA nephropathy is the monitoring of kidney function through determination of proteinuria, serum creatinine, and eGFR. These are all downstream indicators of the disease rather than upstream (in serum) biomarkers that are directly linked to the pathogenesis of the disease. After diagnosis, patients with IgA nephropathy (and their nephrologists) are left in the dark as to whether their prescribed treatment is having any effect on the causes of the disease. They can only wait to see if the rate of decline in kidney function stays the same, improves, or further declines.

In patients with IgA nephropathy, kidneys are damaged as innocent bystanders to an autoimmune disease, as evidenced by two key observations in kidney transplantation: IgA nephropathy frequently recurs in allografts, and IgA deposits clear from kidneys from donors with subclinical IgA nephropathy shortly after transplantation into recipients with non-IgA nephropathy kidney diseases. (Floege, J., Recurrent IgA nephropathy after renal transplantation. Semin. Nephrol., 2004. 24(3): p. 287-91); Silva, F. G., et al., Disappearance of glomerular mesangial IgA deposits after renal allograft transplantation. Transplantation, 1982. 33: p. 214-216). In IgA nephropathy, IgA immunodeposits are enriched for galactose-deficient IgA1 (Gd-IgA1) and the corresponding IgG autoantibodies (IgG-AA). Studies of the glycosylation abnormalities of IgA1 highlighted the key role for Gd-IgA1 in IgA nephropathy.

Research laboratories studying glycosylation abnormalities of IgA1 have found that a potential biomarker for IgA nephropathy is serum Gd-IgA1 levels. Quantitative lectin-based ELISA tests have shown that blood levels of Gd-IgA1 are higher in patients with IgA nephropathy than in healthy controls and in patients with other kidney diseases. (Moldoveanu, Z., et al., Patients with IgA nephropathy have increased serum galactose-deficient IgA1 levels. Kidney Int., 2007). Serum Gd-IgA1 levels also have prognostic value. Higher serum Gd-IgA1 levels are associated with a faster decline in kidney function. (Zhao, N., et al., The level of galactose-deficient IgA1 in the sera of patients with IgA nephropathy is associated with disease progression. Kidney Int, 2012. 82(7): p. 790-6.) The O-glycosylation of IgA1 does not lead to a single modified glycoform of IgA1 but instead produces a mixed population of differentially O-glycosylated IgA1 molecules. (Hiki, Y., et al., Mass spectrometry proves under-O-glycosylation of glomerular IgA1 in IgA nephropathy. Kidney Int., 2001. 59: p. 1077-1085; Allen, A. C., et al., Mesangial IgA1 in IgA nephropathy exhibits aberrant O-glycosylation: Observations in three patients. Kidney Int., 2001. 60: p. 969-973; Renfrow, M. B., et al., Determination of aberrant O-glycosylation in the IgA1 hinge region by electron capture dissociation Fourier transform-ion cyclotron resonance mass spectrometry. J. Biol. Chem., 2005. 280: p. 19136-19145.) In IgA nephropathy, some of these variably O-glycosylated IgA1 molecules, specifically those with galactose deficiency, become recognized as autoantigens by IgG autoantibodies. Gd-IgA1 has been demonstrated to be a disease-specific biomarker for IgA nephropathy. (Yanagawa, H., et al., A Panel of Serum Biomarkers Differenctiates IgA Nephropathy from Other Renal Diseases. PLoS One, 2014, 9(5): p. e9801).

Prior art methods for quantifying Gd-IgA1 include the KM55 antibody assay. This assay employs a rat monoclonal antibody raised against a synthetic IgA1 hinge-region glycopeptide. The synthetic glycopeptide used as the immunogen contained only terminal GalNAc monosaccharide residues, presented at positions and in conformations that may differ from those found in naturally occurring Gd-IgA1. However, IgA1 in serum is a mixture of differentially O-glycosylated proteoforms ranging from 3-6 O-glycans that are each composed of 1 to 4 monosaccharides. Biosynthesis of the clustered O-glycans in IgA1 is a complex process including multiple glycosyltransferases adding monosaccharides in concert. The IgA1 O-glycans are in such close proximity in the IgA1 hinge region, a sialylated O-glycan at one amino acid site can block the galactosylation of an adjacent site. (Takahashi, K., et al., Enzymatic sialylation of IgA1 O-glycans: implications for studies of IgA nephropathy. PLoS One, 2014. 9(2): p. e99026). Thus, a monoclonal antibody such as KM55 that was developed with a synthetic IgA1 glycopeptide that only included monosaccharide GalNAc residues has the potential to be limited and to underrepresent the existing Gd-IgA1 population in a given sample. Accordingly, there remains a need for diagnostic methods and compositions that provide enhanced sensitivity and the ability to detect a more comprehensive spectrum of galactose-deficient IgA1 proteoforms as biomarkers for IgA nephropathy.

### SUMMARY OF THE INVENTION

Provided are methods of diagnosing IgA nephropathy in a human subject. Provided are methods of diagnosing IgA nephropathy (Berger's Disease). Provided are methods of diagnosing IgA vasculitis (Henoch-Schonlein purpura (HSP/HSPN)). Provided are methods of screening potential kidney donors based on a levels of galactose-deficient IgA1.

Provided are methods of determining a level of galactose-deficient IgA1 in the subject. Provided are methods comprising obtaining a biological sample from the subject and using lectin specific for galactose-deficient IgA1 in an assay to detect galactose-deficient IgA1 in the subject.

Provided is a system for diagnosing IgA nephropathy in a human patient. Provided is an assay kit for diagnosing IgA nephropathy in a human patient. In some embodiments, an assay kit for measuring one or more galactose-deficient proteoforms of IgA1 (GalD Assay) in a patient is provided. In some embodiments, methods of preparation and validating quality control (QCs) samples for an assay kit for one or more galactose-deficient proteoforms of IgA1 are provided.

In some embodiments, methods of standardizing one or more batches of lectin and/or biotinylated HPA (or HAA) lectin are provided. In some embodiments, methods of standardizing one or more batches of a biomarker, peptide or protein for use in an assay are provided. In some embodiments methods of monitoring the levels galactose-deficient IgA1 in the context of treatment are provided.

In some embodiments, methods of distinguishing subjects with IgA nephropathy are provided. In some embodiments, methods of determining the severity of IgA nephropathy are provided. In some embodiments, methods of quantifying galactose-deficient IgA1 are provided. In some embodiments, methods of receiving and handling patient samples are provided.

### BRIEF DESCRIPTION OF THE DRAWINGS

The foregoing and other objects, features, and advantages of the invention are apparent from the following detailed description taken in conjunction with the accompanying drawings in which like parts are given like reference numerals and, wherein:

FIG. 1A-D depicts human IgA1 and its hinge-region O-glycans.

FIG. 2 depicts a graph showing serum decline in kidney function relative to serum Gd-IgA1 levels.

FIG. 3 depicts a four parameter standard log curve in accordance with embodiments of the invention.

FIG. 4 depicts standardization of lectin binding activity relative to an IgA1 reference protein

The images in the drawings are simplified for illustrative purposes and are not depicted to scale. Within the descriptions of the figures, similar elements are provided similar names and reference numerals as those of the previous figure(s). The specific numerals assigned to the elements are provided solely to aid in the description and are not meant to imply any limitations (structural or functional) on the invention.

The appended drawings illustrate exemplary configurations of the invention and, as such, should not be considered as limiting the scope of the invention that may admit to other equally effective configurations. It is contemplated that features of one configuration may be beneficially incorporated in other configurations without further recitation.

### DETAILED DESCRIPTION

The embodiments of the disclosure will be best understood by reference to the Figures, wherein like parts are designated by like numerals throughout. It will be readily understood that the components, as generally described and illustrated in the Figures herein, could be arranged and designed in a wide variety of different configurations or be entirely separate. Thus, the following more detailed description of the embodiments of the systems and methods of the disclosure as represented in the Figures is not intended to limit the scope of the disclosure, as claimed, but is merely representative of possible embodiments of the disclosure.

The following description sets forth numerous embodiments and parameters. It should be recognized, however, that such description is not intended as a limitation on the scope of the present invention but is instead provided as a description of exemplary embodiments. Various modifications to the examples described will be readily apparent to those of ordinary skill in the art, and the general principles defined may be applied to other examples and applications without departing from the spirit and scope of the invention. Thus, the present invention is not intended to be limited to the examples described herein but is to be accorded a scope consistent with the claims.

### Definitions

As used herein, “Galactose-deficient IgA1” or “Gd-IgA1” refers to immunoglobulin A1 molecules that exhibit aberrant O-glycosylation in the hinge region, characterized by O-glycan chains that lack galactose residues and terminate with N-acetylgalactosamine (GalNAc) monosaccharides. Gd-IgA1 represents a mixed population of differentially O-glycosylated IgA1 proteoforms where one or more O-glycan sites contain only terminal GalNAc residues rather than complete galactosylated structures. In patients with IgA nephropathy, Gd-IgA1 serves as an autoantigen that is recognized by IgG or other autoantibodies, leading to the formation of pathogenic circulating immune complexes. Gd-IgA1 may also be referred to as “aberrantly glycosylated IgA1” (Ag-IgA1).

As used herein, “Accessible Terminal GalNAc” refers to N-acetylgalactosamine monosaccharides that represent the terminal sugar residues on incompletely glycosylated O-glycan chains of IgA1. These terminal GalNAc residues are present when the normal O-glycosylation process is incomplete, resulting in O-glycan chains that lack the typical galactose and sialic acid additions. Terminal GalNAc residues are also known as Tn antigens and are specifically recognized by lectins such as Helix aspersa agglutinin (HAA) and Helix pomatia agglutinin (HPA), which form the basis for lectin-based detection assays.

As used herein, “Lectin binding activity” refers to the functional capacity of a lectin to specifically recognize and bind to target carbohydrate structures, as distinct from the mere concentration or quantity of lectin present in a solution. Lectin binding activity is a measure of the biological effectiveness of the lectin reagent in detecting specific glycan structures and may vary between different batches or sources of lectin even when present at identical concentrations. This activity is validated through comparative assays measuring optical density responses to ensure consistent and reproducible detection of target glycoproteins across different lectin preparations.

As used herein, “IgA1 hinge region” refers to the specific amino acid sequence region of the IgA1 heavy chain that connects the Fab and Fc portions of the immunoglobulin molecule and contains multiple serine and threonine residues that serve as attachment sites for O-linked glycans. This hinge region is characterized by its flexibility and high density of O-glycosylation sites, typically containing 3-6 clustered O-glycan attachment points. The hinge region is also the site where IgA-specific proteases can cleave the IgA1 molecule, yielding different Fab and Fc fragments.

As used herein, “IgA1 hinge region O-glycosylation” refers to the post-translational modification process, wherein carbohydrate structures are covalently attached to serine and threonine residues within the IgA1 hinge region through O-linkages. This process involves the sequential addition of monosaccharides by multiple glycosyltransferases, beginning with N-acetylgalactosamine (GalNAc) followed by galactose (Gal) and potentially sialic acid. For purposes of this specification, sialic acid and neuraminic acid are interchangeable carbohydrate structures.

As used herein, “IgA1 hinge region O-glycosylation mixtures” refers to the heterogeneous population of differentially O-glycosylated IgA1 molecules present in human serum, where individual IgA1 molecules contain varying combinations of complete and incomplete O-glycan structures across the multiple glycosylation sites in the hinge region. These mixtures range from fully galactosylated proteoforms to those with varying degrees of galactose deficiency (containing 1, 2, 3, or more sites with terminal GalNAc residues) intermixed with fully glycosylated sites. The composition of these mixtures differs between healthy individuals and patients with IgA nephropathy, with the latter showing elevated levels of proteoforms containing 2-3 sites of galactose deficiency combined with other fully galactosylated sites.

As used herein, “Parallelism” is a measure of similarity between the dose-response curve of a test sample and the standard curve in an ELISA assay, confirming that the endogenous analyte in a sample is detected by the antibodies in the same way as the purified standard. It is tested during assay validation by serially diluting samples with high levels of endogenous analyte and comparing their response to the standard curve. Parallelism ensures that the sample matrix does not alter antibody binding and that the standard can be used to accurately quantify the analyte.

As used herein, “Non-terminal GalNac O-glycans” refers to O-linked glycan structures where N-acetylgalactosamine (GalNAc) is present as the core monosaccharide but is extended by additional sugar residues such as galactose and/or sialic acid. These structures represent complete or fully elaborated O-glycans in which the GalNAc is not exposed as the terminal sugar. Non-terminal GalNAc structures are not accessible to binding by GalNAc-specific lectins when the GalNAc residue is masked by galactose or sialic acid additions.

IgAN, also known as Berger's disease, is the most common primary glomerulonephritis and an important cause of kidney failure. IgA nephropathy is a mesangioproliferative glomerular disease defined by characteristic IgA1 mesangial deposits. These mesangial deposits likely originate from circulating immune complexes that contain IgA1 with Galactose (Gal)-deficient O-glycans (Gd-IgA1, the autoantigen) that are bound by IgG autoantibodies and other serum proteins. The established pathogenesis model of IgA nephropathy enabled the pharmaceutical industry to start developing and testing treatments for the disease. However, only secondary markers (e.g., proteinuria and estimated glomerular filtration rate [eGFR]) are currently used as the endpoints, adding to the time and cost of clinical trials. Thus, clinical-grade tests that assess primary causative markers are urgently needed. The present invention can be used for an N-acetylgalactosamine (GalNAc)-specific lectin-based Gd-IgA1 assay that detects accessible terminal GalNAc (i.e., Galactose-deficient) O-glycans on IgA1 in serum and will allow scaled production of lectin-based ELISA that measures Gd-Iga1 levels. This can only be done with standardized lectin binding activity and a standard for IgA1 galactose deficiency.

IgA nephropathy is the most frequent cause of kidney failure among Asians and the leading cause of glomerulonephritis among young Caucasians. Currently, evaluation of a kidney biopsy is necessary for diagnosis, with routine immunofluorescence microscopy revealing immunodeposits with predominant or co-dominant IgA, with variable presence of IgG and/or IgM, and usually with complement C3 co-deposits. The exact prevalence of IgA nephropathy is not known, but the frequency of clinically silent disease may be high. IgA nephropathy frequently manifests with episodes of macroscopic hematuria or acute nephritic syndrome that coincide with mucosal infections. The incidence of IgA nephropathy peaks in young adults in the 2nd and 3rd decades of life. IgA nephropathy reduces life expectancy by as much as 10 years. Moreover, in the absence of disease-specific treatment, within 20 years after the diagnostic kidney biopsy, about 20-40% of patients progress to kidney failure.

Diagnosing kidney disease is a complicated path that most often starts with either a standard urinalysis or the occurrence of cloudy (proteinuria) and/or bloody urine (hematuria). The subsequent evaluation process usually includes measurement of the serum creatinine and determination of the estimated glomerular filtration rate (eGFR) and review of the patient's pertinent medical history and demographics to develop the differential-diagnosis decision tree. For most of the possible outcomes, the process culminates in a kidney biopsy. However, the procedure entails significant risk for bleeding complications that may require transfusion, embolization of the bleeding vessel, removal of the bleeding biopsied kidney, and, rarely, death. As a consequence, biopsy is sometimes delayed to determine if the proteinuria or hematuria persists. Also, there is the possibility that the biopsy yields inconclusive results if an insufficient specimen is obtained. Kidney biopsy is the only means to diagnose IgA nephropathy because there is no other disease-specific test for IgA nephropathy. There is a clear unmet need for a non-invasive means to diagnose and monitor this chronic kidney condition through disease-specific biomarkers and not solely a decline in kidney function.

The inherent risks associated with kidney biopsy often delay the diagnosis of IgA nephropathy. In many cases this includes irreversible kidney damage. Thus, providing a diagnostic test that could eventually be deployed as a screening tool for IgA nephropathy in its early stages will inherently change the clinical course of patients, as IgA nephropathy will be suspected earlier or IgA nephropathy could be eliminated earlier as the cause of the hematuria/proteinuria. The scalable IgA nephropathy-specific biomarker assay discussed herein will provide a method to clearly identify IgA nephropathy patients early in the differential-diagnosis decision tree. This advancement will save time and money as it will decrease clinician time and the costs of laboratory testing or radiological imaging procedures. Additionally, once diagnosed, the disclosed biomarker assays will 1) help stratify patients at risk of disease progression, 2) serve to monitor the disease activity in IgA nephropathy patients, 3) determine if treatments are lowering these causal components of disease, and 4) for patients needing a kidney transplant, allow screening of potential living donors for IgA nephropathy.

Further, the lack of direct biomarker assays for IgA nephropathy has limited development of therapeutics that can directly target the root cause of the disease. Current clinical trials have been limited to evaluating conventional kidney function readouts (e.g., eGFR, proteinuria). Given that improvement of kidney function may require 6 to 9 months once the circulating load of disease-inducing immune complexes has decreased, availability of direct marker tests that can monitor the blood levels of the molecules that cause IgA nephropathy (Gd-IgA1 and IgG-autoantibodies) would significantly decrease the duration needed to assess the efficacy of therapeutics tested in clinical trials.

However, implementation of such direct biomarker assays requires the establishment of a standardized reference for both lectin binding activity and IgA1 galactose deficiency. Variability in lectin source, glycan presentation, and binding kinetics can result in inconsistent detection of galactose-deficient IgA1, thereby limiting inter-laboratory comparability and assay reproducibility. Accordingly, the invention provides for the use of calibrated lectin reagents and a defined Gd-IgA1 reference standard. The use of such standardized materials ensures that the lectin-IgA1 interaction is quantitatively comparable between assay lots and across testing sites, thereby enabling reproducible quantification of Gd-IgA1.

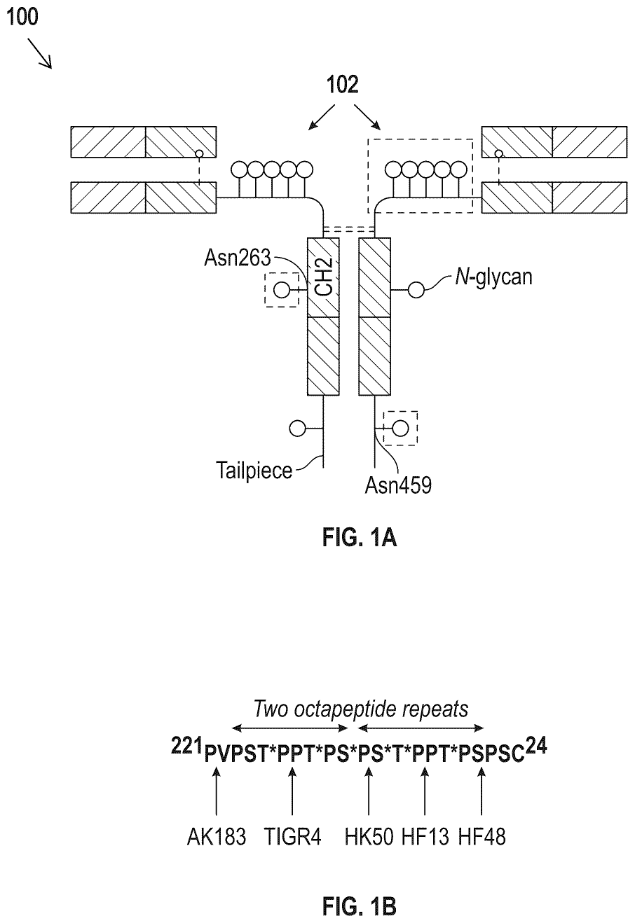

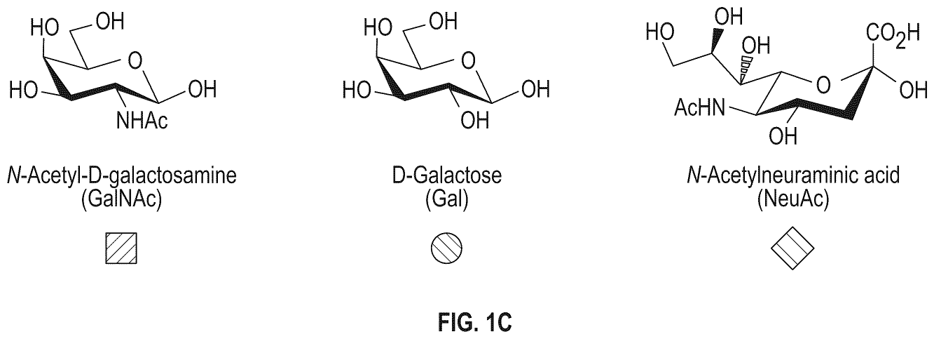

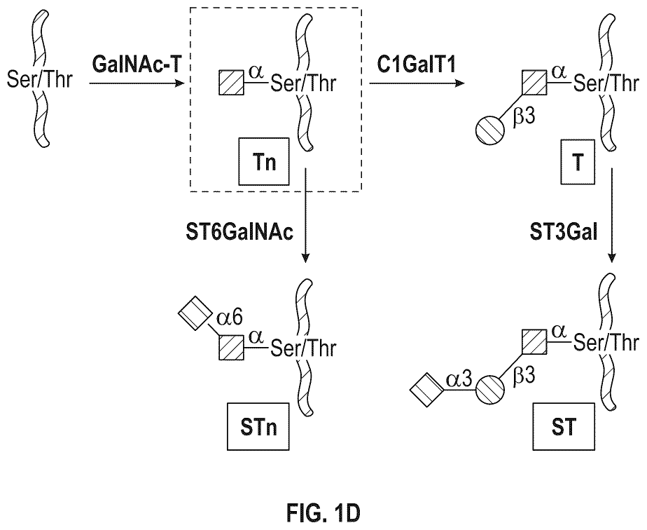

IgA1 mesangial deposits likely originate from circulating immune complexes that contain IgA1 with Galactose (Gal)-deficient O-glycans (Gd-IgA1, the autoantigen) that are bound by IgG autoantibodies. As shown in FIGS. 1A-D, IgA1 heavy chain 100 has, in addition to two N-glycans, a hinge-region 102 and 3-6 clustered O-glycans, consisting of GalNAc, Gal, and sialic acid. IgA-specific proteases cleave IgA1 at the sites (AK183, TIGR4, HK50, HF13, HF48), yielding different Fab and Fc fragments (FIG. 1). Defects in enzymes in IgA1-producing cells in IgA nephropathy patients result in elevated production of Gd-IgA1 (FIG. 1D). IgA1 with Tn antigen (terminal GalNAc) is pathogenic, as it is recognized by IgG autoantibodies. Other glycoforms (T, STn, and ST antigens) are not bound by IgG autoantibodies.

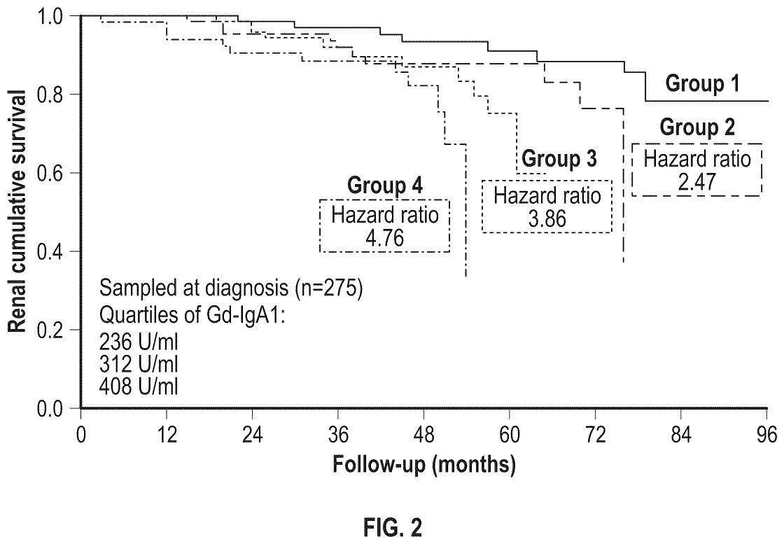

IgA1 O-glycosylation defects are universally detected in IgA nephropathy and are associated with disease progression. Comprehensive studies of the glycosylation abnormalities of IgA1 offered a potential biomarker for IgA nephropathy: serum Gd-IgA1 levels. Quantitative lectin-based ELISA tests have shown that blood levels of Gd-IgA1 are higher in patients with IgA nephropathy than in healthy controls and patients with other kidney diseases in European, Asian, and African-American cohorts. Additionally, the serum Gd-IgA1 level has prognostic value. Higher amounts of serum Gd-IgA1 are associated with a faster decline in kidney function, defined as kidney failure. See FIG. 2. The O-glycosylation of IgA1 does not lead to a single modified glycoform of IgA1 but instead produces a mixed population of differentially O-glycosylated IgA1 molecules. In IgA nephropathy, some of these variably O-glycosylated IgA1 molecules, specifically those with Gal deficiency, become recognized as autoantigens by IgG autoantibodies.

As shown in FIG. 2, elevated serum Gd-IgA1 level predicts faster disease progression in IgA nephropathy patients. Group 1 depicts the lowest quartile and Group 4 depicts the highest quartile of Gd-IgA1 levels.

IgA1 has native levels of Gd-IgA1 that naturally exist in normal human serum. In patients with IgA nephropathy, there is a shift in the composition of O-glycan structures that results in a higher presentation of Gd-IgA1 forms. Native populations of IgA1 have mixtures of proteoforms with 3-6 O-glycan chains. Some IgA1 proteoforms have complete galactosylation, while others have mixtures of complete O-glycan chains combined with one or two sites with only GalNAc (galactose-deficiency) or sialylated-GalNAc. Patients with IgA nephropathy have higher levels of galactose deficient O-glycosylated proteoforms.

The foundational studies used to identify Gd-IgA1 as a key component of the pathogenesis of IgA nephropathy were conducted with a lectin-based ELISA test. Lectins are carbohydrate-binding proteins that are highly specific for sugar groups. In lectin-based ELISAs, lectins are used instead of antibodies to detect glycoproteins. Lectins that recognize galactose-deficiency (terminal GalNAc) on IgA1 (Gd-IgA1) are distinct from monoclonal antibodies generated with a synthetic hinge-region glycopeptide.

The reason for under-representation of Gd-IgA1 levels has to do with the fact that IgA1 in serum is a mixture of differentially O-glycosylated proteoforms ranging from 3-6 O-glycans that are each composed of 1 to 4 monosaccharides as shown in FIG. 1. Biosynthesis of the clustered O-glycans in IgA1 is a complex process including multiple glycosyltransferases adding monosaccharides in concert. The IgA1 O-glycans are in such close proximity in the IgA1 hinge region, a sialylated O-glycan at one amino acid site can block the galactosylation of an adjacent site. Thus, a monoclonal antibody such as KM55 that was developed with a synthetic IgA1 glycopeptide that only included monosaccharide GalNAc residues, has the potential to be limited and to underrepresent the existing Gd-IgA1 population in a given sample.

Lectins, are proteins that recognize specific carbohydrate groups. Research has demonstrated that the snail lectins Helix aspersa agglutinin (HAA) and Helix pomatia agglutinin (HPA) bind individual GalNAc residues. They also demonstrated that these lectins can distinguish IgA1 myeloma proteins that have differing degrees of monosaccharide GalNAc O-glycan that are intermixed with di- and trisaccharides (fully galactosylated) O-glycans. While lectin-based ELISA studies have been conducted, the values reported for individual studies are difficult to compare across time, different patient cohorts, and across different laboratories who have developed their own assays due, at least in part, to the variability in lectin binding activities based on batch to batch purification differences. There is a need to have a standardized lectin-based ELISA for measurement of Gd-IgA1 that relies on a calibrated Gd-IgA1 level in serum and other biological fluids.

The present invention include methods for measuring and analyzing Gd-IgA1, methods for diagnosing the presence and severity of IgA nephropathy, an assay kit for the quantification of Gd-IgA1, methods for quantification of Gd-IgA1, an assay kit for the diagnosis of IgA nephropathy, a serum standard comprised of one or more of native IgA1, enzyme modified IgA1, recombinant IgA1, and methods of validating two or more batches of lectin reagent. In some embodiments, the systems and methods disclosed herein may be used to diagnose one or more of IgA nephropathy (Berger's Disease) and IgA vasculitis (Henoch-Schonlein purpura (HSP/HSPN)). Provided are methods of screening potential kidney donors based on levels of galactose-deficient IgA1 and monitoring levels of Gd-IgA1 during the course of patient treatment. The use of GalNAc-specific lectin as a standardized reagent is a novel aspect of this invention. The method of standardization of different batches of lectin as a reagent, so that it provides reproducible and predictable results, is a novel aspect of this invention. The specificity of the lectin for GalNAc allows recognition of multiple sites of Gal-deficiency that exist in serum populations of IgA1 proteoforms.

In some embodiments, the assay employs serial dilutions of a patient sample to ensure accurate quantification across a dynamic range of galactose-deficient IgA1 concentrations. However, in certain embodiments, it has been found that a single dilution may be sufficient to produce a testable sample for greater than 90% of patient specimens, thereby simplifying the workflow and reducing reagent use without compromising analytical performance.

### Methods for Determining Gal-Deficient IgA1 in a Biological Sample.

In one aspect, methods are provided for detecting galactose-deficient IgA1 in a biological sample. The methods generally comprise isolating IgA1 antibodies present in the biological sample, contacting the isolated IgA1 with a standardized lectin reagent that recognizes galactose-deficient glycoforms, and measuring the resulting signal to quantify Gd-IgA1 levels. In some embodiments, the method comprises:

(a) Isolating the IgA1 antibodies present in the biological sample by contacting the biological sample with an IgA1-specific capture reagent to create IgA1 complexes; (b) Contacting the IgA1 complexes with a detection reagent to form sandwich IgA1 complexes, wherein the detection reagent is configured to bind to more than one type of galactose-deficient IgA1 proteoforms; (c) measuring a signal resulting from the presence of the sandwiched IgA1 complexes.

The biological sample may be any sample that contains or is suspected to contain IgA1 antibodies. Suitable biological samples include, but are not limited to, serum, plasma, urine, other biological fluids, cell samples, cell secretions, cell isolates, secreted IgA1 from isolated B-cells that originated from IgAN patients or other individuals. and other cell preparations. In certain embodiments, the biological sample is a serum sample obtained from a human subject. The biological sample may be used directly or may be diluted prior to analysis.

The first step of the method comprises isolating IgA1 antibodies present in the biological sample by contacting the biological sample with an IgA1-specific capture reagent to create IgA1 complexes, wherein the IgA1 complexes may comprise the capture reagent and one Gal-deficient IgA proteoform bound together. The IgA1-specific capture reagent comprises binding molecules that selectively bind to IgA1 antibodies. Any suitable IgA1-specific capture reagent can be used. In various non-limiting embodiments, the capture reagent comprises antibodies, antibody fragments, aptamers, or other binding molecules that specifically recognize IgA1. In one specific embodiment, the capture reagent comprises an anti-human IgA antibody that is specific for the IgA heavy chain. Such antibodies are commercially available from various vendors. In certain embodiments, the capture reagent comprises an AffiniPure F(ab′)2 fragment of goat IgG anti-human IgA that is specific for the gamma chain Fc-fragment. The use of F(ab′)2 fragments can reduce non-specific binding and improve assay specificity.

The capture reagent may be bound to a surface to facilitate isolation of the IgA1 antibodies from the biological sample. Any suitable surface can be used, including but not limited to the wells of a microtiter plate, magnetic or paramagnetic beads, agarose beads, glass, cellulose, polyacrylamide, nylon, polystyrene, polypropylene supports, and filtration media. In one specific embodiment, the capture reagent is adsorbed to the surface of wells in a microtiter plate, such as a 96-well plate, a 384-well plate or any other suitable well-plate.

The capture reagent may be immobilized on the surface by any suitable method. In one embodiment, the capture reagent is adsorbed to the surface by passive adsorption. For example, the capture reagent may be diluted in a coating buffer and incubated in contact with the surface for a time and under conditions sufficient to allow adsorption. In one non-limiting embodiment, the capture reagent is diluted to a concentration of between about 1 g/mL to about 10 g/mL in a coating buffer. In a further embodiment, the capture reagent is diluted to a concentration of about 2.5 g/mL in a phosphate buffered saline (PBS) solution containing sodium azide as a preservative. The diluted capture reagent may be incubated with the surface overnight at a temperature of about 4° C.

Following immobilization of the capture reagent, the surface may be blocked to prevent non-specific binding. Any suitable blocking agent can be used, including but not limited to bovine serum albumin (BSA), casein, dried milk, fish gelatin, and commercial blocking buffers. In one specific embodiment, the surface is blocked with a solution comprising about 1% BSA in PBS containing a detergent such as Tween 20 (PBS-T). The blocking may be performed for a time of about 30 minutes to about 4 hours at room temperature. In a further embodiment, the blocking is performed for about 2 hours at room temperature. After blocking, excess blocking solution may be removed and the blocked surface may be stored until use. In one embodiment, the blocked surface is stored at about −20° C. for up to about 6 months.

The biological sample is contacted with the immobilized capture reagent for a time and under conditions sufficient to allow IgA1 antibodies in the sample to bind to the capture reagent and form IgA1 complexes. The biological sample may be diluted prior to contacting with the capture reagent. In various embodiments, the biological sample is diluted by a factor of about 100-fold to about 10,000-fold. In one embodiment, the biological sample is diluted about 2,000-fold in a reagent buffer comprising BSA and a detergent. The dilution may be performed in one step or in multiple serial dilution steps. In one embodiment, the sample is first diluted about 100-fold by adding about 5 μL of sample to about 495 μL of reagent buffer. A portion of this first dilution (about 20 μL) is then added to about 380 μL of reagent buffer to produce a final dilution of about 2,000-fold. Other dilution schemes producing final dilutions in the range of about 500-fold to about 5,000-fold may also be used. The diluted sample is incubated with the immobilized capture reagent for about 30 minutes to about 2 hours at room temperature. In one embodiment, the incubation is performed for about 1 hour at room temperature (about 20-25° C.).

Following incubation, unbound material may be removed by washing. The washing may be performed with any suitable wash buffer, such as PBS or PBS.

After isolation of the IgA1 antibodies, the IgA1 complexes are contacted with a detection reagent to form sandwich complexes (IgA1-detector complexes), wherein the sandwich complexes comprises the capture reagent bound to a IgA1 proteoform and the IgA1 proteoform also bound to the detection reagent. The detection reagent is configured to bind to galactose-deficient IgA1 proteoforms. Importantly, the detection reagent is capable of binding to more than one type of galactose-deficient IgA1 proteoform, allowing for detection of certain heterogeneous Gd-IgA1 population present in biological samples. Any suitable detection reagent that recognizes galactose-deficient glycans can be used. In various embodiments, the detection reagent comprises a lectin, an antibody, an antibody fragment, an aptamer, or other binding molecule that selectively binds to galactose-deficient glycans. In a preferred embodiment, the detection reagent comprises a lectin.

Lectins are a widely used reagent across many different biotechnology fields to monitor relative levels of specific glycosylation structures. However, it is well known that lectins produced in vitro and isolated from natural sources have a wide range of activity variability due to some of the isolated lectin being inactive after purification. Thus, lectins have rarely been used as reagents in standardized assay formats that report absolute concentration values. This is where the submitted invention defines a means of standardizing the lectin binding activity of any produced preparation of lectin for detection of accessible terminal GalNAc on IgA1. It also defines the means to do so for any given lectin activity. Suitable lectins for use as detection reagents include, but are not limited to, Helix pomatia agglutinin (HPA), Helix aspersa agglutinin (HAA), peanut agglutinin (PNA), and Vicia villosa agglutinin (VVA). In one embodiment, the detection reagent comprises HPA lectin, which binds specifically to N-acetylgalactosamine (GalNAc) residues that are exposed when accessible terminal galactose is absent.

The detection reagent may comprise a detectable label or moiety that allows for measurement of binding. Any suitable detectable label can be used, including but not limited to fluorescent labels, enzyme labels, radioactive labels, chemiluminescent labels, colorimetric labels, and the like. In one embodiment, the detection reagent comprises a lectin that is conjugated to biotin. The biotin label allows for subsequent detection using avidin or streptavidin conjugated to an enzyme or other detectable moiety.

The detection reagent is contacted with the IgA1 complexes for a time and under conditions sufficient to allow binding of the detection reagent to galactose-deficient IgA1. In one embodiment, the detection reagent is diluted in a reagent buffer and added to the IgA1 complexes. The dilution of the detection reagent may be optimized based on the specific detection reagent used and the desired sensitivity and dynamic range of the assay. In one non-limiting embodiment using biotin-labeled HPA lectin, a lyophilized lectin is reconstituted in water and then diluted in a reagent buffer comprising BSA and a detergent. For example, a lyophilized lectin pellet may be reconstituted in about 100 μL of water and then added to about 12 mL of reagent buffer for use in analyzing a 96-well plate. The detection reagent is incubated with the IgA1 complexes for about 30 minutes to about 2 hours at room temperature. In one embodiment, the incubation is performed for about 1 hour at room temperature. Following incubation, unbound detection reagent may be removed by washing as described above.

Following binding of the detection reagent, a measurable signal is generated and detected. The method of signal generation and detection depends on the type of detectable label present on the detection reagent.

In embodiments where the detection reagent comprises a biotin label, the method further comprises contacting the sandwich complexes with an enzyme conjugate to form a conjugated complex. The enzyme conjugate comprises avidin or streptavidin conjugated to an enzyme. Suitable enzymes include, but are not limited to, horseradish peroxidase (HRP), alkaline phosphatase, β-galactosidase, and glucose oxidase. In one embodiment, the enzyme conjugate comprises avidin conjugated to peroxidase.

The enzyme conjugate is contacted with the sandwich complexes for a time and under conditions sufficient to allow binding. In one embodiment, the enzyme conjugate is diluted in a reagent buffer. For example, the enzyme conjugate may be diluted to a dilution factor of about 1,000-fold to about 3,000-fold. In an embodiment, the enzyme conjugate is diluted about 2,000-fold in reagent buffer (e.g., about 6 μL per 12 mL of buffer for one 96-well plate).

The enzyme conjugate is incubated with the sandwich complexes for about 30 minutes to about 2 hours at room temperature. In one embodiment, the incubation is performed for about 1 hour at room temperature. Following incubation, unbound enzyme conjugate is removed by washing.

After removal of unbound enzyme conjugate, the conjugated complex is contacted with a substrate for the enzyme to produce a measurable signal. The substrate is selected based on the enzyme present in the enzyme conjugate. For peroxidase enzymes, suitable substrates include, but are not limited to, 3,3′,5,5′-tetramethylbenzidine (TMB), 2,2′-azino-bis(3-ethylbenzothiazoline-6-sulphonic acid) (ABTS), o-phenylenediamine (OPD), and 3,3′-diaminobenzidine (DAB). In one embodiment, the substrate is TMB, which produces a colorimetric reaction when oxidized by peroxidase. The TMB substrate may be diluted prior to use. In one embodiment, a 100% TMB buffer is diluted 1:1 with deionized water to produce a 50% TMB buffer. About 100 μL of the diluted TMB buffer is added to each well and incubated for about 15 minutes to about 45 minutes at room temperature. In one embodiment, the incubation is performed for about 30 minutes at room temperature.

The colorimetric reaction is stopped by addition of a stop solution. Suitable stop solutions include acidic solutions such as sulfuric acid or hydrochloric acid solutions. In one embodiment, about 100 μL of stop solution is added to each well.

The measurable signal (e.g., the colorimetric reaction if Biotion/TMB is used) is then measured. For colorimetric reactions, the signal is typically measured as optical density (OD) at a specific wavelength using a microplate reader. Other means of signal detection may be used depending on the type of detectable label used in the assay. For TMB substrate, the optical density is typically measured at about 450 nm, but any suitable wavelength known in the art may be chosen. The measurement is preferably performed within about 30 minutes of stopping the reaction.

The measured signal correlates with the amount of accessible terminal galactose-deficient IgA1 present in the biological sample. Higher signals indicate higher levels of Gd-IgA1. The concentration of Gd-IgA1 in the sample can be determined by comparison to a standard curve as described below.

In one embodiment, the methods further comprise generating a standard curve for quantification of Gd-IgA1 concentration. The standard curve is generated using serial dilutions of a standard sample containing a known concentration of Gd-IgA1.

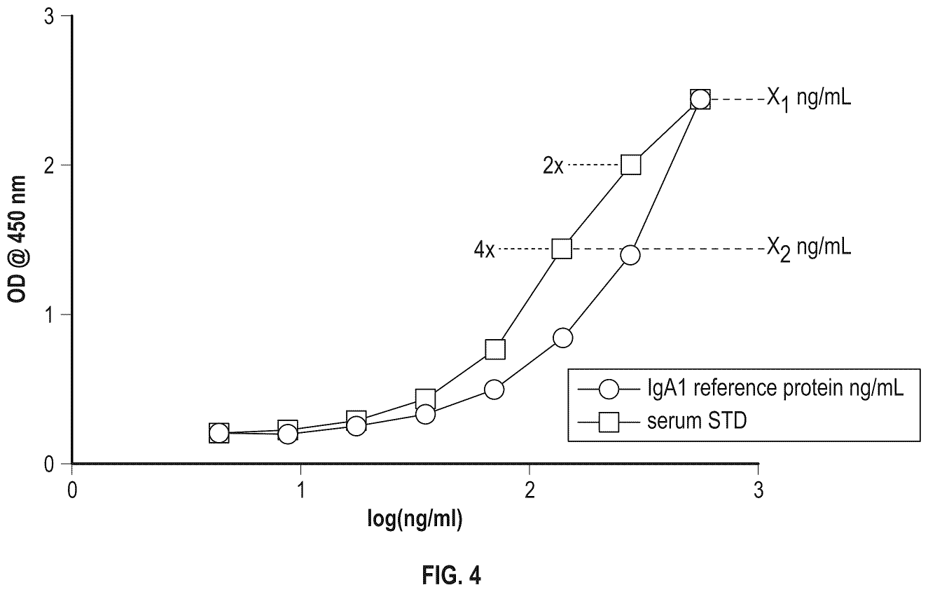

The reference standard (also “calibration standard”) sample may be a stable, engineered sample with a known and stable concentration of Gd-IgA1. The reference standard may be benchmarked relative to a well-characterized Gd-IgA1 reference material with known IgA1 concentration. As demonstrated in FIG. 4, the reference standard exhibits parallelism with both the well-characterized Gd-IgA1 reference material as well as non-modified human serum samples when combined with a standardized HPA lectin. Parallelism, in the context of immunoassays, is a measure of similarity between the dose-response curve of a test sample and the standard curve, confirming that the analyte in a sample is detected by the detection reagent in the same manner as the purified standard. The demonstration of parallelism between the reference standard and both the well-characterized Gd-IgA1 reference material and natural serum samples validates that the reference standard accurately represents the range of Gd-IgA1 proteoforms present in patient samples. This parallelism ensures that when patient samples are quantified using the reference standard curve, the measured values accurately reflect the true concentration of Gd-IgA1 in the sample, rather than being artificially skewed by differences in how the detection reagent interacts with different forms of the analyte.

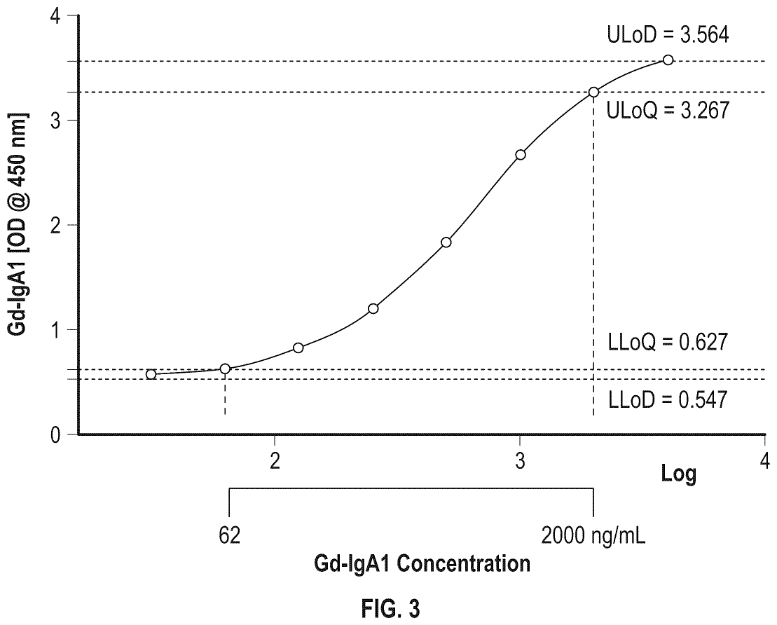

In one embodiment, the reference standard is prepared by selecting a serum sample from a cohort of samples and matching its concentration to purified Gd-IgA1. The serum sample is aliquoted, lyophilized, and stored at about −20° C. until use. The dilution of the standard, as well as the lyophilization and dehydration further contribute to the stability of the standard. The long-term stability of the reference standard is critical for ensuring reproducibility across different assay runs, different laboratories. To prepare the standard curve, the standard sample is reconstituted (if lyophilized) and serially diluted to produce a series of standards with decreasing concentrations. Any suitable dilution factor may be used. In one embodiment, a 2-fold serial dilution is performed to generate about 6 to about 10 different standard concentrations. In one embodiment, 7 standards are prepared by performing 2-fold serial dilutions. An example of standard curve prepared in accordance with some embodiments of the invention is shown in FIG. 3.

For example, a stock standard (STD 0) may be prepared at a concentration of about 4,000 ng/mL. This stock is then serially diluted 2-fold to produce STD 1 (2,000 ng/mL), STD 2 (1,000 ng/mL), STD 3 (500 ng/mL), STD 4 (250 ng/mL), STD 5 (125 ng/mL), STD 6 (62.5 ng/mL), and STD 7 (31.25 ng/mL).

The standards are analyzed in the same manner as the biological samples, and the resulting measured signals (such as optical density values) are plotted against the known concentrations. A regression curve is fit to the data using an appropriate mathematical model. Suitable regression models include, but are not limited to, linear regression, logarithmic regression, polynomial regression, and four-parameter logistic (4PL) regression.

In one specific embodiment, a four-parameter logistic curve is used to fit the standard curve data, however, other models may be used. The 4PL model is particularly suitable for immunoassays and provides good fit across a wide dynamic range. The quality of the fit is assessed by calculating the coefficient of determination (R²). In one embodiment, an R² value of at least about 0.95 is considered acceptable. In a further embodiment, an R² value of at least about 0.98 is required for acceptable assay performance. The concentration of Gd-IgA1 in unknown samples is determined by comparing the measured signal from the sample to the standard curve and interpolating the concentration. The interpolated concentration is then multiplied by any dilution factors to determine the concentration in the original undiluted sample. In various embodiments, the methods include analysis of quality control (QC) samples in addition to standards and test samples. QC samples are samples with known concentrations of Gd-IgA1 that are analyzed alongside test samples to verify assay performance.

The QC samples may be prepared in a manner similar to the standard samples. In one embodiment, multiple QC samples with different concentrations spanning the measurement range are prepared. For example, three QC samples (QC1, QC2, and QC3) with high, medium, and low concentrations, respectively, may be prepared.

### Reference Standard

In another aspect, reference standards (also “calibration standard”) are provided for use in assays for measuring galactose-deficient IgA1. The reference standard sample is a stable, engineered sample with a known and stable concentration of Gd-IgA1. The reference standard is benchmarked relative to a well-characterized Gd-IgA1 reference material with known IgA1 concentration. As demonstrated in FIG. 4, the reference standard exhibits parallelism with both the well-characterized Gd-IgA1 reference material as well as non-modified human serum samples when combined with a standardized HPA lectin. Importantly, the reference standard is stable over time as opposed to non-stable natural standards.

In various embodiments, the reference standard is a novel serum sample comprising one or more of naturally occurring Gd-IgA1 purified from a biological source, enzymatically modified IgA1 that has been treated to produce galactose-deficient glycans, recombinantly produced IgA1 that is enzymatically modified to present Gd-IgA1, recombinantly produced Gd-IgA1, or synthetically produced IgA1, IgA1 heavy chain, or IgA1 hinge region with extended domains on N- or C-terminal portions. In some embodiments, the reference standard is a novel serum sample comprising one or more of enzymatically modified IgA1 that has been treated to produce galactose-deficient glycans, recombinantly produced IgA1 that is enzymatically modified to present Gd-IgA1, recombinantly produced Gd-IgA1, or synthetically produced IgA1, IgA1 heavy chain, or IgA1 hinge region with extended domains on N- or C-terminal portions. The well-characterized Gd-IgA1 reference material has a measurable relative abundance of terminal-GalNAc O-glycans of 20-80% compared to non-terminal-GalNAc O-glycan.

In some embodiments, the reference standard partially or wholly comprises enzymatically modified IgA1 that has been treated to produce galactose-deficient glycans. In some embodiments the IgA1 has been enzymatically modified with neuraminidase. The enzymatic treatment of the standard helps provide a consistent matrix and increase the stability of the serum standard over time. In some embodiments, the reference standard is enzymatically treated serum with up to 50% recombinantly produced Gd-IgA1.

In one embodiment, the reference standard is prepared by selecting a serum sample from a cohort of samples and matching its concentration to purified Gd-IgA1. The serum sample is aliquoted, lyophilized, and stored at about −20° C. until use. The dilution of the standard, as well as the lyophilization and dehydration further contribute to the stability of the standard.

In some embodiments, assay quality control standards may be derived from the reference standard discussed herein.

The calibration standard may be used to generate a standard curve that defines the quantitative range of an assay. As shown in FIG. 3, a series of serial dilutions of one embodiment of the calibration standard (STD 0 through STD 7) are prepared and analyzed to produce a dose-response curve. The optical density values obtained from each standard concentration are plotted against the corresponding standard concentrations to generate a standard curve.

### General Applicability of Standardization and Validation Methods to Other Reagents, Biomarkers, Peptides, and Proteins

While the methods described herein are exemplified through the standardization and validation of lectin reagents for detecting galactose-deficient IgA1, the underlying principles and procedures are broadly applicable to the standardization and validation of various binding reagents used to detect diverse biomarkers, peptides, and proteins in analytical and diagnostic assays.

The methodology disclosed, comparing the binding activity or functional performance of a test batch of reagent to a well-characterized reference batch using appropriate calibration standards, defined acceptance criteria, and iterative refinement, provides a general framework applicable across multiple assay platforms and target analytes. The core principles remain consistent regardless of the specific reagent type or molecular target: establish a reference standard with known properties, prepare test materials at multiple defined concentrations or dilutions, perform comparative binding or activity assays, measure appropriate signals, and iteratively adjust concentrations until predetermined acceptance criteria are met.

### Methods of Validating Sources of Detectable Lectin

In some embodiments, a method of standardizing a test batch of lectin to a reference batch of lectin for use as a reagent is provided. The method may comprise: providing an amount of a reference batch of lectin with known binding activity; providing an amount of a test batch of lectin; providing a calibration standard to calibrate the activity of the lectin molecule from any source for detection of its natural glycan target in a biological sample; preparing at least a portion of the test batch of lectin into one or more aliquots of known concentrations; performing a test to assess the binding activity of the one or more aliquots of the test batch lectin and the reference batch lectin to the calibration standard; and selecting the one or more known concentrations of the test batch lectin with binding activity most similar to the reference batch of lectin for use as a reagent.

In some embodiments, the similarity of the one or more known concentrations of test batch lectin and reference lectin is determined by plotting a measured signal correlating to binding activity for the test batch and reference batch and comparing the plots. In some embodiments, the measured signal is the optical density of a produced colorimetric reaction, however other suitable methods of measuring bind activity, such as the use and measurement of fluorescent labels, enzyme labels, radioactive labels, chemiluminescent labels, colorimetric labels, may be selected as well. In some embodiments, selecting the one or more known concentrations of the test batch lectin with binding activity most similar to the reference batch of lectin is based on acceptance criteria, wherein the acceptance criteria comprises the test batch lectin having a plotted measured signal with an analytical recovery within 15% of the plotted measured signal of the reference batch, wherein analytical recovery is defined as the actual value divided by the measurement and the product multiplied by 100% (measurement/actual×100%). In some embodiments, the acceptance criteria for analytical recovery may be set at various thresholds depending on the precision requirements of the assay and its intended clinical or research application. Suitable acceptance criteria include, but are not limited to, analytical recovery within 5%, 6%, 7%, 8%, 9%, 10%, 11%, 12%, 13%, 14% 15% of the reference batch's measured signal.

If the one or more known concentrations of test batch lectin do not have a plotted measured signal that satisfies the acceptance criteria, the method may comprise additional iterative steps. These steps include selecting the known concentration of test batch lectin that has a plotted measured signal closest to satisfying the acceptance criteria and creating a subsequent set of further known concentrations from said known concentration, wherein the subsequent range of further known concentrations may be: a range having the best-fit known concentration as a midpoint; a range having the best-fit known concentration as a midpoint, wherein the range is narrower than the range of a previous test; or a range, wherein an endpoint of the range is the best-fit known concentration. The method then repeats the testing steps using the reference batch of lectin and further known concentrations of the test batch of lectin until a known concentration or further known concentration of the test batch of lectin produces a plotted measured signal that satisfies the acceptance criteria.

In some embodiments, performance qualification (validation) process for lectin or biotinylated HPA (or HAA) lectin is provided. The process involves standardizing lectin binding activity relative to reference lectin using a molecular characterized IgA1 standard. In some embodiments, the performance qualification process of a batch of lectin or biotinylated HPA (or HAA) lectin is conducted using a modified ELISA assay. In some embodiments, the performance qualification process of a batch of lectin or biotinylated HPA (or HAA) lectin is conducted using any form of test for binding activity. In some embodiments, the performance qualification process of a batch of lectin or biotinylated HPA (or HAA) lectin is conducted using any form of assay for glycosylated proteins, synthetic glycosylated peptides, purified glycosylated peptides, or glycosylated amino acids.

In some embodiments, the biological sample comprises one or more of serum, urine, other biological fluid, a cell sample, a cell secretion, cell isolate, or other cell preparations. In some embodiments, the calibration standard is a well-characterized standard of the natural glycan presented in a biologically relevant molecule. In some embodiments, the calibration standard comprises one or more of naturally occurring Gd-IgA1, enzymatically modified IgA1 (that allows presentation of Gd-IgA1), recombinantly produced IgA1 that is enzymatically modified to present Gd-IgA1, or recombinantly produced Gd-IgA1. In some embodiments, the known concentrations of test batch of lectin comprise a concentration of 1% to 500% of the concentration of the reference batch of lectin.

In some embodiments, assay quality control standards may be derived from the standardized lectin or methods discussed herein.

In some embodiments, a qualification process comprises a comparison of a batch of lectin (and reference lectin (QUALIFIED) in the run either on the same well plate or using different well plates. Whether a comparison of new biotinylated lectin (lectin-BIO) and reference lectin (QUALIFIED) occurs on the same well plate or different well plates depends on the source of new lectin. In some embodiments, the comparison of new lectin-BIO and reference lectin may occur on the same day. In some embodiments, the comparison of new lectin-BIO batch and reference lectin may occur on a single plate. In some embodiments, the comparison of new lectin-BIO and reference lectin may occur one or more separate plates from the same batch. The disclosed steps may be performed manually or through automation.

In some embodiments, the process for validating lots of biotinylated lectin obtained from a new source/provider may comprise the following steps: preparing one or more serial dilutions of a biomarker, peptide or target protein (such as Gd-IgA1) standard starting with a predetermined concentration. In some embodiments, the biomarker, peptide or target protein used in the standard and serial dilutions is one or more of Gd-IgA1, glycosylated proteins, synthetic glycosylated peptides, purified glycosylated peptides, or glycosylated amino acids. The use of different biomarkers, peptide or target proteins may create different accessible glycosylated patterns that would provide unique validation characteristics for a given lectin. In some embodiments, the lectin validation procedure uses two or more 2× serial dilutions of a biomarker, peptide or target protein standard starting with a predetermined concentration (such as 1, 2, 3 or 4, 5, 6, 7 or more μg/ml) and two or more biomarker, peptide or target protein QC samples. In some embodiments, the lectin validation procedure, uses seven 2× serial dilutions of Gd-IgA1 standard starting with concentration of 2 μg/ml and eight Gd-IgA1 QC samples. In some embodiments, the smallest container of new lyophilized lectin has to be diluted in water. In some embodiments, the smallest container of new lyophilized lectin has to be diluted in water to the concentration of 1 mg/mL, aliquoted to separate tubes containing 100 μg/100 μl (100%), and frozen at −20° C. An additional step of the lectin validation process comprises preparing a reference plate or arrangement, wherein the reference plate or arrangement is appropriate to compare the reactivity of one or more batches and/or concentrations of lectin with one or more dilutions of one a biomarker standard. In some embodiments, the additional step of preparing a reference plate or arrangement comprises preparing a reference plate or arrangement with a new source of biotinylated lectin according to the plate map shown in Table 1, wherein STD means “Standard”, QC means “Quality Control” and BLN means “Blank”.

TABLE 1

| 1 | 2 | 3 | 4 | 5 | 6 | 7 | 8 | 9 | 10 | 11 | 12 |
|---|---|---|---|---|---|---|---|---|---|---|---|
| STD1 | STD1 | QC1 | QC1 | BLN | BLN | BLN | BLN | BLN | BLN | BLN | BLN |
| STD2 | STD2 | QC2 | QC2 | BLN | BLN | BLN | BLN | BLN | BLN | BLN | BLN |
| STD3 | STD3 | QC3 | QC3 | BLN | BLN | BLN | BLN | BLN | BLN | BLN | BLN |
| STD4 | STD4 | QC4 | QC4 | BLN | BLN | BLN | BLN | BLN | BLN | BLN | BLN |
| STD5 | STD5 | QC5 | QC5 | BLN | BLN | BLN | BLN | BLN | BLN | BLN | BLN |
| STD6 | STD6 | QC6 | QC6 | BLN | BLN | BLN | BLN | BLN | BLN | BLN | BLN |
| STD7 | STD7 | QC7 | QC7 | BLN | BLN | BLN | BLN | BLN | BLN | BLN | BLN |
| BLN | BLN | QC8 | QC8 | BLN | BLN | BLN | BLN | BLN | BLN | BLN | BLN |

In some specific embodiments, an additional step of the lectin validation process comprises preparing a test plate or arrangement using different concentrations of new lectin-BIO. In some embodiments, the test plate or arrangement uses concentrations of 100 μg (100%), 90 μg (90%), and 80 μg (80%). However, other concentrations may be used depending on the qualities of the reference of test lectin. As discussed above, the concentrations of the test lectin may be anywhere from 1% to 500% of the concentration for the reference lectin. In some embodiments, the test plate or arrangement uses any concentration(s) appropriate of test lectin. In some embodiments, the test plate/arrangement is arranged as shown in Table 2, wherein STD means “Standard”, QC means “Quality Control” and BLN means “Blank”.

TABLE 2

| 100% | | | | 90% | | | | 80% | | | |
|---|---|---|---|---|---|---|---|---|---|---|---|
| 1 | 2 | 3 | 4 | 5 | 6 | 7 | 8 | 9 | 10 | 11 | 12 |
| STD1 | STD1 | QC1 | QC1 | STD1 | STD1 | QC1 | QC1 | STD1 | STD1 | QC1 | QC1 |
| STD2 | STD2 | QC2 | QC2 | STD2 | STD2 | QC2 | QC2 | STD2 | STD2 | QC2 | QC2 |
| STD3 | STD3 | QC3 | QC3 | STD3 | STD3 | QC3 | QC3 | STD3 | STD3 | QC3 | QC3 |
| STD4 | STD4 | QC4 | QC4 | STD4 | STD4 | QC4 | QC4 | STD4 | STD4 | QC4 | QC4 |
| STD5 | STD5 | QC5 | QC5 | STD5 | STD5 | QC5 | QC5 | STD5 | STD5 | QC5 | QC5 |
| STD6 | STD6 | QC6 | QC6 | STD6 | STD6 | QC6 | QC6 | STD6 | STD6 | QC6 | QC6 |
| STD7 | STD7 | QC7 | QC7 | STD7 | STD7 | QC7 | QC7 | STD7 | STD7 | QC7 | QC7 |
| BLN | BLN | QC8 | QC8 | BLN | BLN | QC8 | QC8 | BLN | BLN | QC8 | QC8 |

An additional step of the lectin validation process may comprise comparing the measured signal (such as optical densities (OD)) of standard, quality controls, and blanks and then comparing the measured signals of the different concentrations for quality controls to find the best match of measured signal between a concentration group of test lectin and the reference lectin, in order to match the different concentrations of test lectin and reference lectin for quality control. For example, if the above concentration range (100-80%) for a test lectin plate did not match the ODs of the reference plate with 70% of lectin, the assay needs to be run again with the lower concentrations of new lectin, for example: 80%/70%/60% and so on, containing the lowest concentration of lectin from the plate run a day before (here 80%), until the ODs value matches the activity of the reference lectin. In some embodiments, a dilution of the reference lectin is used. In some embodiments, the new lectin concentration group is considered standardized when one or more acceptance criteria are met. The acceptance criteria may include the variance between measured signal values of one or more new lectin concentration groups and reference lectin group is below a predetermined acceptable level.

In some embodiments, acceptance criteria may include a less than 3% variance in measured signal between a new lectin concentration group and the reference lectin group. In various embodiments, the level of variance may be another amount that is suitable for the given reagent, such as anywhere from 1-15% variance. In some embodiments, acceptance criteria may include a new lectin concentration group having an acceptable level of variance in measured signal relative to a reference lectin group produced across at least 90% of replicates of the new lectin concentration group. In some embodiments, the acceptance criteria may allow for acceptable variance across a different amount of replicates, such as anywhere from 75-99% of replicates. In some embodiments, the validation criteria includes: if at least 90% of replicates of the new lectin concentration group have variance within target levels of variance (for example <3%), then the remaining 10% of replicates of the new lectin concentration group do not have a variance in measured signal greater than a predetermined value relative to the reference lectin group. The acceptable variance for the non-acceptable replicates may be less than 10% or from 1-20%. In some embodiments, the target variance in measured signals for validation of a concentration of new lectin batch relative to a reference batch of lectin is less than three percent (<3%) variance in 9 out of 10 replicates, with the outlier replicate having no more than 10% deviation from the target.

In some embodiments, the method of the lectin validation may further comprise iterating one or more of the aforementioned steps with groups of new lectin concentrations having incrementally tighter ranges of concentrations or different ranges until one or more validation criteria are met. For example, if after comparing the measured signals of the 80%/70%/60% new lectin concentrations, the variance in measured signals of the 70% concentration group is the lowest variance of the three concentration groups relative to the reference lectin but not below a target level, the validation test is run again use tighter concentrations, such as 75%/70%/65%, and the measured signals compared again until the validation criteria are met by a concentration group. Upon satisfaction of one or more of or all of the validation criteria, a batch of lectin is deemed fit for use in an assay or other testing method.

In some embodiments, the process for validating lots of lectin or new biotinylated lectin batch(es) obtained from the same source/provider as a previous batch may comprise the following steps: first, preparing one or more serial dilutions of Gd-IgA1 (or other biomarker, target molecule, or protein) standard starting with a predetermined concentration. In some embodiments, the lectin validation procedure uses seven 2× serial dilutions of Gd-IgA1 (or other biomarker, target molecule or protein) standard starting with concentration of 2 μg/ml and eight Gd-IgA1 (or other biomarker, target molecule or protein) QC samples. Other suitable numbers of dilutions or concentrations may be used. For example, the initial biomarker standard may be 1-200 μg/ml. An additional step of the lectin validation process comprises preparing a reference arrangement of reference lectin and a test arrangement of new lectin on the same plate according to the arrangement shown in Table 3 (wherein STD means “Standard”, QC means “Quality Control” and BLN means “Blank”).

TABLE 3

| 1 | 2 | 3 | 4 | 5 | 6 | 7 | 8 | 9 | 10 | 11 | 12 |
|---|---|---|---|---|---|---|---|---|---|---|---|
| STD1 | STD1 | QC1 | QC1 | BLN | BLN | STD1 | STD1 | QC1 | QC1 | BLN | BLN |
| STD2 | STD2 | QC2 | QC2 | BLN | BLN | STD2 | STD2 | QC2 | QC2 | BLN | BLN |
| STD3 | STD3 | QC3 | QC3 | BLN | BLN | STD3 | STD3 | QC3 | QC3 | BLN | BLN |
| STD4 | STD4 | QC4 | QC4 | BLN | BLN | STD4 | STD4 | QC4 | QC4 | BLN | BLN |
| STD5 | STD5 | QC5 | QC5 | BLN | BLN | STD5 | STD5 | QC5 | QC5 | BLN | BLN |
| STD6 | STD6 | QC6 | QC6 | BLN | BLN | STD6 | STD6 | QC6 | QC6 | BLN | BLN |
| STD7 | STD7 | QC7 | QC7 | BLN | BLN | STD7 | STD7 | QC7 | QC7 | BLN | BLN |
| BLN | BLN | QC8 | QC8 | BLN | BLN | BLN | BLN | QC8 | QC8 | BLN | BLN |

| | |
|---|---|
| Ref. Lectin: 70% | New Lectin: 70% |

In some embodiments, the process may further comprise the additional step of comparing the measured signals of standard, quality controls, and blanks. Alternative measured parameters may be used instead of optical density. If concentrations do not match the measured signals obtained from the reference lectin, one or more additional runs are performed using a 2-plate process. The 2-plate process comprises preparing a reference plate or arrangement, with the reference batch of biotinylated lectin (or lectin) according the plate map shown in Table 4, wherein STD means “Standard”, QC means “Quality Control” and BLN means “Blank”.

TABLE 4

| 1 | 2 | 3 | 4 | 5 | 6 | 7 | 8 | 9 | 10 | 11 | 12 |
|---|---|---|---|---|---|---|---|---|---|---|---|
| STD1 | STD1 | QC1 | QC1 | BLN | BLN | BLN | BLN | BLN | BLN | BLN | BLN |
| STD2 | STD2 | QC2 | QC2 | BLN | BLN | BLN | BLN | BLN | BLN | BLN | BLN |
| STD3 | STD3 | QC3 | QC3 | BLN | BLN | BLN | BLN | BLN | BLN | BLN | BLN |
| STD4 | STD4 | QC4 | QC4 | BLN | BLN | BLN | BLN | BLN | BLN | BLN | BLN |
| STD5 | STD5 | QC5 | QC5 | BLN | BLN | BLN | BLN | BLN | BLN | BLN | BLN |
| STD6 | STD6 | QC6 | QC6 | BLN | BLN | BLN | BLN | BLN | BLN | BLN | BLN |
| STD7 | STD7 | QC7 | QC7 | BLN | BLN | BLN | BLN | BLN | BLN | BLN | BLN |
| BLN | BLN | QC8 | QC8 | BLN | BLN | BLN | BLN | BLN | BLN | BLN | BLN |

In some embodiments, the process may further comprise the additional step of preparing a test plate or arrangement using different concentrations of new lectin for comparison relative to the reference plate/arrangement. In some embodiments, lectin concentrations of the test plate depend on the results of the previously performed single plate test. For example, if on the single plate test the ODs on the new order lectin from Provider A were higher than ODs of the reference lectin-BIG from Provider A, the test plate includes 90%/80%/70% concentrations. If the ODs from new order lectin were lower than 70% of the reference lectin, the test plate should include 70%/60%/50% concentrations. In both scenarios, the reference plate uses a 70% concentration. The test plate/arrangement is arranged as shown in Table 5, wherein STD means “Standard”, QC means “Quality Control” and BLN means “Blank”.

TABLE 5

| 90% | | | | 80% | | | | 70% | | | |
|---|---|---|---|---|---|---|---|---|---|---|---|
| 1 | 2 | 3 | 4 | 5 | 6 | 7 | 8 | 9 | 10 | 11 | 12 |
| STD1 | STD1 | QC1 | QC1 | STD1 | STD1 | QC1 | QC1 | STD1 | STD1 | QC1 | QC1 |
| STD2 | STD2 | QC2 | QC2 | STD2 | STD2 | QC2 | QC2 | STD2 | STD2 | QC2 | QC2 |
| STD3 | STD3 | QC3 | QC3 | STD3 | STD3 | QC3 | QC3 | STD3 | STD3 | QC3 | QC3 |
| STD4 | STD4 | QC4 | QC4 | STD4 | STD4 | QC4 | QC4 | STD4 | STD4 | QC4 | QC4 |
| STD5 | STD5 | QC5 | QC5 | STD5 | STD5 | QC5 | QC5 | STD5 | STD5 | QC5 | QC5 |
| STD6 | STD6 | QC6 | QC6 | STD6 | STD6 | QC6 | QC6 | STD6 | STD6 | QC6 | QC6 |
| STD7 | STD7 | QC7 | QC7 | STD7 | STD7 | QC7 | QC7 | STD7 | STD7 | QC7 | QC7 |
| BLN | BLN | QC8 | QC8 | BLN | BLN | QC8 | QC8 | BLN | BLN | QC8 | QC8 |

In some embodiments a dilution of the reference lectin is used. In some embodiments, the new lectin concentration group is considered validated when one or more validation criteria are met. The validation criteria may include whether or not the variance between measured signal values of one or more new lectin concentration groups and the reference lectin group is below a predetermined acceptable level.

In some embodiments, validation criteria includes a less than 3% variance in measured signals between a new lectin concentration group and the reference lectin group. In some embodiments, validation criteria includes a new lectin concentration group having an acceptable level of variance in measured signal relative to a reference lectin group produced across at least 90% of replicates of the new lectin concentration group. In some embodiments, the validation criteria includes if at least 90% of replicates of the new lectin concentration group have variance within target levels of variance (for example <3%), then the remaining 10% of replicates of the new lectin concentration group do not have a variance in measured signal greater than 10% relative to the reference lectin group. In some embodiments, the target variance in measured signals for validation of a concentration of new lectin batch relative to a reference batch of lectin is less than three percent (<3%) variance in 9 out of 10 replicates, with the outlier replicate having no more than 10% deviation from the target.

In some embodiments, the method of the lectin validation may further comprise repeating one or more of the aforementioned steps with groups of new lectin concentrations having incrementally tighter ranges of concentrations until one or more validation criteria are met. For example, if after comparing the measured signals of the 80%/70%/60% new lectin concentrations, the variance in measured signals of the 70% concentration group is the lowest variance of the three concentration groups relative to the reference lectin, but the variance is not below a target level, the validation test is run again use tighter concentrations, such as 75%/70%/65%, and the measured signals compared again until the validation criteria are met by a concentration group. Upon satisfaction of one or more validation criteria, a batch of lectin is deemed fit for use in an assay or other testing method.

A method of assay quantification range determination is disclosed. The method comprises the following steps: First, calculating the mean, standard deviation (SD), and coefficient of variation (% CV) from two replicates of the standard curves. Next, accepting run results only if successful curve fit is demonstrated using a four-parameter logistic sigmoidal curve. In some embodiments, all curves must maintain R² value of >0.98, when compared to determine key assay range characteristics (background will be calculated from the average maximum value observed in each plate blank region. These will determine background mean, SD, and from these the LLOD (Lower Limit of Detection)). Next, LLOQ, minimum and maximum asymptote/ULOD (if observed) are calculated. Accuracy will be expressed as percentage of analytical recovery (% AR) and percentage of relative error (% RE) wherein % AR=(Actual value/Measurement)×100% and % RE=((Measurement−Actual value)/Actual value)×100%. Precision will be expressed as the percent coefficient of variation (% CV), the ratio of the standard deviation to the mean at analyte level, wherein % CV=SD/Mean×100%. The intra-assay accuracy and precision is accepted when the CV for all tested QC samples is ≤15%. In various embodiments, other criteria may be used for example, accuracy and or precision values of anywhere from 1-25%. Accuracy acceptance criteria for samples within the quantitative range are ±10% AR and ±20% at LLOQ, ULOQ. In various embodiments, other criteria may be used for example, samples within the quantitative range can be anywhere from ±20% AR and ±35% at LLOQ, ULOQ. The total allowable analytical error should be ≤30%, except ULOQ where can be ≤40%. Upon acceptance, a batch of lectin is deemed fit for use in an assay or other testing method.

The methods disclosed herein for standardizing reagents are not limited to biotinylated HPA or HAA lectins used for detecting galactose-deficient IgA1. Rather, the standardization methods described may be broadly applied to validate and standardize any binding reagent for use in detecting target biomarkers, peptides, or proteins in analytical assays. Further standardization methods may used to validate and standardize quality control standards and calibration standards.

The disclosed standardization methodology provides a general framework applicable to any reagent-based assay system where batch-to-batch consistency is critical for reproducible results. The fundamental principle—comparing the binding activity of a test batch of reagent to a reference batch using appropriate calibration standards and acceptance criteria-remains consistent regardless of the specific reagent or target analyte.

### Use in Patient Care of IgA Nephropathy

The methods and assays disclosed herein provide multiple clinical applications for the diagnosis, prognosis, risk stratification, treatment selection, and monitoring of IgA nephropathy, kidney disease and/or renal failure in patients. These applications address critical needs in the management of IgA nephropathy by providing direct measurement of a causative biomarker rather than relying solely on downstream indicators of kidney function decline.

A method of diagnosing IgA nephropathy in a patient based on the amount of Gal-deficient IgA1 present in the blood, urine, and/or serum of the patient is disclosed. The method may comprise a number of steps; firstly, determining a patient's Gal-deficient IgA1 levels. In some embodiments, a patient's Gal-deficient IgA1 levels are determined by using a N-acetylgalactosamine (GalNAc)-specific lectin-based Gd-IgA1 assay. In some embodiments, the step of determining a patient's Gal-deficient IgA1 levels incorporates one or more of the steps of administering a Gal-deficient IgA1 assay and/or quantifying Gal-deficient IgA1 in a patient of this specification.

The method may further comprise the step of comparing the patient's Gal-deficient IgA1 levels relative to established reference ranges for healthy controls and for patients with confirmed IgA nephropathy. The method may further comprise making one of the following determinations based on the comparison of the patient's Gal-deficient IgA1 levels: attributing a low risk of IgA nephropathy to patients with Gal-deficient IgA1 levels that are below the mean for healthy controls; attributing an increased likelihood of IgA nephropathy to patients with Gal-deficient IgA1 levels that are above the mean for healthy controls and within the range observed in IgA nephropathy patients; or recommending further observation and serial monitoring for patients with Gal-deficient IgA1 levels that are above the mean for healthy controls but below the typical range for confirmed IgA nephropathy.

In some embodiments, the quantified Gd-IgA1 levels are used for prognostic assessment and risk stratification of patients with diagnosed or suspected IgA nephropathy. Elevated serum Gd-IgA1 levels are associated with faster rates of kidney function decline and increased risk of progression to end-stage renal disease. The methods disclosed herein enable clinicians to stratify patients into risk categories based on their Gd-IgA1 levels, thereby informing clinical decision-making regarding the intensity of monitoring and the aggressiveness of therapeutic intervention.

In some embodiments, patients are stratified into risk categories such as low risk, intermediate risk, high risk, or very high risk based on their quantified Gd-IgA1 levels relative to established thresholds. For example, patients with Gd-IgA1 levels in the highest quartile may be classified as high risk for rapid disease progression, while those in lower quartiles may be classified as having more indolent disease. This risk stratification allows for personalized management strategies tailored to each patient's individual risk profile.

In some embodiments, the prognostic assessment further comprises determining the likelihood of progression to end-stage renal disease within a defined time period (such as 1 year, 5 years, 10 years, or 20 years) based on the measured Gd-IgA1 levels or based on the measured Gd-IgA1 levels in combination with other clinical parameters such as degree of proteinuria, estimated glomerular filtration rate (eGFR), etc.

The quantified Gd-IgA1 levels may be used to guide treatment selection and inform therapeutic decisions. In some embodiments, patients with elevated Gd-IgA1 levels, particularly those in the highest risk categories, may be prioritized for more aggressive treatment interventions.

In some embodiments, the method comprises recommending initiation of disease-modifying therapy for patients with Gd-IgA1 levels above a predetermined threshold that indicates high risk of disease progression. In some embodiments, the method comprises recommending continuation, modification, or escalation of existing therapy based on serial measurements of Gd-IgA1 levels that demonstrate persistent elevation despite treatment.

In some embodiments, patients with lower Gd-IgA1 levels may be managed with more conservative approaches without the need for more aggressive immunosuppressive interventions and their associated risks and side effects.

The disclosed methods and inventions may be used to monitor a patient's Gal-deficient IgA1 levels during the course of the patient's disease and treatment. Serial measurements of Gd-IgA1 levels overtime provide an assessment of disease activity and enable monitoring of treatment response in a manner that is more directly related to the underlying disease mechanism than traditional markers.

In some embodiments, the method comprises obtaining serial measurements of Gd-IgA1 levels at regular intervals (such as every 3 months, every 6 months, or annually) to track changes in disease activity over time. Decreasing Gd-IgA1 levels during treatment may indicate a favorable therapeutic response and suggest that the treatment is effectively targeting the underlying pathogenic mechanisms of the disease. Conversely, persistently elevated or increasing Gd-IgA1 levels despite treatment may indicate inadequate therapeutic response and prompt consideration of treatment modification or escalation.

In some embodiments, the methods disclosed herein are used to assess disease progression in patients with established IgA nephropathy. The method may comprise determining whether a patient's disease is stable, slowly progressive, or rapidly progressive based on changes in Gd-IgA1 levels over time in combination with changes in kidney function markers.

In some embodiments, increasing Gd-IgA1 levels over time, particularly when accompanied by declining eGFR or worsening proteinuria, indicate active disease progression and may prompt more aggressive therapeutic intervention. In some embodiments, stable or decreasing Gd-IgA1 levels, particularly in patients receiving treatment, indicate disease stabilization and suggest that the current therapeutic approach is appropriate.

In some embodiments, the rate of change in Gd-IgA1 levels is calculated and used as a prognostic indicator. For example, a rapid increase in Gd-IgA1 levels over a short time period (such as 6-12 months) may indicate aggressive disease requiring urgent therapeutic intensification, while gradual changes may indicate more indolent disease progression.

In some embodiments, the Gal-deficient IgA1 assay, the embodiments of the method of administering the assay and/or the embodiments of the method of quantification of Gal-deficient IgA1 using the assay may be incorporated into diagnostic and assessment algorithms for IgA nephropathy or other kidney diseases, such as those prescribed in medical journals and clinical practice guidelines. For example, the Gal-deficient IgA1 assay and associated methods may be incorporated into the assessment and management plans for IgA nephropathy provided in Kidney Disease: Improving Global Outcomes (KDIGO) guidelines or other authoritative clinical practice guidelines.

In some embodiments, the methods are used as part of a comprehensive diagnostic workup for patients presenting with hematuria and/or proteinuria to determine whether IgA nephropathy is the likely underlying diagnosis, potentially obviating the need for kidney biopsy in some cases or supporting the decision to proceed with biopsy in others. In some embodiments, the methods are incorporated into post-diagnosis assessment protocols to guide initial risk stratification and treatment planning.

The embodiments of the assay, the embodiments of the method of administering the assay and/or the embodiments of the method of quantification of Gal-deficient IgA1 using the assay may be used in clinical decision-making related to kidney transplantation. In some embodiments, the methods are used to screen potential living kidney donors for IgA nephropathy or identifying donors with elevated Gd-IgA1 levels who may be at risk for having or developing IgA nephropathy and may therefore be unsuitable as kidney donors.

In some embodiments, the methods are used to assess the risk of disease recurrence in kidney transplant recipients with a history of IgA nephropathy. Elevated Gd-IgA1 levels in transplant recipients may indicate increased risk of IgA nephropathy recurrence in the allograft and may prompt closer monitoring or consideration of interventions to prevent or delay recurrence.

In one embodiment, a method of analyzing a biological sample to determine a risk of IgA nephropathy may comprise: (a) obtaining a biological sample from a subject; (b) determining an amount of one or more galactose-deficient IgA1 proteoforms in the sample; and (c) diagnosing a likelihood of IgA nephropathy in a subject, wherein an increased amount of one or more galactose-deficient IgA1 proteoforms in the subject's sample as compared to a control indicates the subject has or is at risk of developing IgA nephropathy. The step of determining an amount of one or more galactose-deficient IgA1 proteoforms may further comprise: (b1) isolating the IgA1 antibodies present in the biological sample by contacting the biological sample with an IgA1-specific capture reagent to create IgA1 complexes; (b2) contacting the IgA1 complexes with a detection reagent to form sandwich complexes, wherein the detection reagent is configured to bind to more than one type of galactose-deficient IgA1 proteoforms; and (b3) measuring a signal resulting from the presence of the sandwich complexes.

In one embodiment, a method of analyzing a biological sample to determine a prognosis of a case of IgA nephropathy may comprise: (a) obtaining a biological sample from a subject; (b) determining an amount of one or more galactose-deficient IgA1 proteoforms in the sample; and (c) determining a prognosis of the IgA nephropathy in a subject based on the amount of one or more galactose-deficient IgA1 proteoforms in the subject's sample as compared to a control. The step of determining an amount of one or more galactose-deficient IgA1 proteoforms may comprise the steps of: (b1) isolating the IgA1 antibodies present in the biological sample by contacting the biological sample with an IgA1-specific capture reagent to create IgA1 complexes; (b2) contacting the IgA1 complexes with a detection reagent to form sandwich complexes, wherein the detection reagent is configured to bind to more than one type of galactose-deficient IgA1 proteoforms; and (b3) measuring a signal resulting from the presence of the sandwich complexes.

In one embodiment, the method of analyzing a biological sample to determine the severity of a case of IgA nephropathy may comprise: (a) obtaining a biological sample from a subject having IgA nephropathy; (b) determining an amount of one or more galactose-deficient IgA1 proteoforms in the sample; (c) determining the severity of the IgA nephropathy in a subject, based on an amount of one or more galactose-deficient IgA1 proteoforms in the subject's sample as compared to a control. The step of determining an amount of one or more galactose-deficient IgA1 proteoforms may further comprise: (b1) isolating the IgA1 antibodies present in the biological sample by contacting the biological sample with an IgA1-specific capture reagent to create IgA1 complexes; (b2) contacting the IgA1 complexes with a detection reagent to form sandwich complexes, wherein the detection reagent is configured to bind to more than one type of galactose-deficient IgA1 proteoforms; and (b3) measuring a signal resulting from the presence of the sandwich complexes. The method of determining the severity of IgA nephropathy may further comprise the step of (d) selecting a treatment method based on the severity of the IgA nephropathy in a subject.

### Biomarker Assay Kit

In some embodiments, a kit comprises a solid support, a capture reagent, and a signal generating material. The kit can also include instructions for using the devices and reagents, handling the sample, and analyzing the data. Further, the kit may be used with a computer system or software to analyze and report the result of the analysis of the biological sample.

The kits can also contain one or more reagents (e.g., solubilization buffers, detergents, washes, or buffers) for processing a biological sample. Any of the kits described herein can also include: buffers, blocking agents, mass spectrometry matrix materials, antibody capture agents, positive control samples, negative control samples, software and information such as protocols, guidance and reference data.

In some embodiments, kits are provided. In some embodiments, an assay kit for quantitative measurement of levels of galactose-deficient IgA1 (Gd-IgA1) in human serum samples is provided. In some embodiments, levels of galactose-deficient IgA1 (Gd-IgA1, aberrantly glycosylated IgA1, Ag-IgA1) in human samples are measured quantitatively over 1 day using an indirect sandwich HPA Lectin ELISA assay, on a previously prepared pre-coated plate.

In some embodiments, the kit may comprise one or more reagents. In some embodiments, the kit may comprise: an IgA capturing antibody, such as anti-human IgA (gamma chain Fc-fragment-specific); Biotin-labeled HPA lectin; 3,3′,5,5′-Tetramethylbenzidine (TMB); Avidin-Peroxidase conjugate (such as ExtrAvidin-Peroxidase); Bovine Serum Albumin (BSA). In some embodiments, the Gd-IgA1 assay kit may further comprise the following reagents and chemicals: Type 1 water; 10× Phosphate Buffer Saline (PBS); Sodium azide (NaN₃); Tween-20 (Polyoxyethylenesorbitan monolaurate); Sulfuric acid (96%); Hydrochloric acid (HCl); Sodium hydroxide (NaOH); Hydrogen Peroxide (H₂O₂).

In some embodiments, the assay kit comprises a well-characterized IgA1 reference standard and a standardized lectin reagent. In some embodiments, the reference standard comprises one of the engineered IgA1 reference standard described herein.

The lectin reagent included in the kit is a GalNAc-specific lectin that has been standardized according to the methods disclosed herein to ensure reproducible binding activity across different batches. In some embodiments, the lectin is biotinylated Helix pomatia agglutinin (HPA) or biotinylated Helix aspersa agglutinin (HAA).

Some embodiments of the Gd-IgA1 assay kit may comprise one or more of the solutions described herein: a 10× coating buffer comprised of 10×PBS/0.1% NaN3 [1 L]; A 1× coating buffer comprised of 1×PBS/0.1% NaN3 [1 L]; A 1×PBS buffer [1 L]; 1×PBS-T buffer [1 L]; 50% TMB; A Stop Solution comprised of 0.1 M sulfuric acid [500 mL]; 10% Tween-20 [50 ml]; The blocking/antibody buffer (complete Reagent Buffer (cRB))—1% BSA/PBS-T [500 mL].

In any of the embodiments of the Gd-IgA1 assay kits or methods disclosed herein, the coating buffer or wash buffer may be substituted with (in the proper amounts and concentrations) any buffered salt solution comprising 50-250 mM salt (such as NaCl, KCl, or a suitable alternative) and 5 to 100 mM buffering agent (e.g. phosphate, tris, HEPES, MES, or a suitable alternative), wherein the pH is 6.2-8.0.

In any of the embodiments of the Gd-IgA1 assay kits or methods disclosed herein any of the detergents listed may be substituted with any other suitable detergent such as triton, tween-20, etc. at concentrations of 0.01%-1.0%. In any of the embodiments of the Gd-IgA1 assay kits or methods disclosed herein, any of the STOP solutions listed may be substituted with any acidic agent sufficient to increase the pH within the assay reaction environments. In any of the embodiments of the Gd-IgA1 assay kits or methods disclosed herein, any of the visualization reagents may be substituted with TMB to change the wavelength of the observation. In any of the embodiments of the Gd-IgA1 assay kits or methods disclosed herein, any of the blocking agents may be substituted with any blocking reagent sufficient to stop non-specific hydrophobic and/or charged association with the reagent well.

The 10× coating buffer may be made by adding 1 g of NaN₃ to 1 L of 10×PBS. The 1× coating buffer may be made by adding by adding 100 mL of 10×PBS/0.1% NaN³ to 900 mL water in a graduated cylinder. A 1×PBS buffer [1 L] may be made by adding 100 mL 10×PBS to 900 mL of water in a graduated cylinder (1× working concentrations: 137 mM NaCl, 2.7 mM KCl, 8 mM Na2HPO4, 2 mM KH2PO4). 1×PBS-T buffer [1 L] may be made by adding 100 mL 10×PBS to 900 mL of water to a gradated cylinder and adding 2.5 mL of Tween-20 to 0.05% final concentration. The 50% TMB may be made by adding 6 mL of water to 15 mL conical tube containing 6 mL of 100% TMB solution. The Stop Solution 0.1 M sulfuric acid [500 mL] may be made by placing 472 mL of water in a 1-liter gradated Pyrex-type storage bottle, adding 27.8 mL of 96% H2SO4 to the bottle and mix it gently. The 10% Tween-20 [50 ml] may be made by transferring 5 ml of Tween-20 to 45 ml water and mixing gently. The blocking/antibody buffer (complete Reagent Buffer (cRB))—1% BSA/PBS-T [500 mL] may be made by weighing out 5 g of BSA and add 400 mL of 1×PBS buffer in a 1-liter beaker placed on a magnetic stirrer and, after the BSA dissolves, bringing the total volume in the container to 500 mL by adding water and adding 2.5 mL of Tween-20 (Polyethylene glycol sorbitan monolaurate, Polyoxyethylenesorbitan monolaurate) to 0.05% final concentration.

Some embodiments of the Gd-IgA1 assay kit may comprise one or more of the materials described herein: 96-well Flat-Bottom medium binding plates; Wypall X60 teri towels; Plastic beakers: 1000 ml, 2000 ml; Weighing boats; Tips 0.1-20 μL; Tips 2-200 μL; Pipette Tips 50-1000 μL; Tips ClipTip Filtered 1250 μL; Tips Finntip 10, 0.2-10 μL; Tips Finntip Flex 300, 30-300 μL; Sterile disposable serological pipets: 10 ml, 20 ml, 50 ml; Graduated cylinders: 500 ml and 1000 ml; Eppendorf single-channel pipettes: P20, P100, P200, P1000; Manual Finnpipette F2 Multichannel pipette (8 channel): 2-10 μl; Electronic Finnpipette Novus Multichannel Pipette 8 channel 30-300 μL; Electronic E1-ClipTip Thermo Scientific Adjustable Multichannel—15 to 1250 μL; Mixer; Analytical balance; BioTek Synergy 4 Microplate Reader; 2° C. to 8° C. refrigerator for storage of sera and reagents; A −20° C. freezer; A −80° C. freezer. The kit may include suitable substitutes.

In some embodiments of administering the GalD Assay, levels of galactose-deficient IgA1 (Gd-IgA1, aberrantly glycosylated IgA1, Ag-IgA1) in human samples are measured quantitatively over 1 day using an indirect sandwich HPA Lectin ELISA assay, on the previously prepared pre-coated plate. In some embodiments, the administration of the GalD Assay generally comprises a first phase: the adsorption of human specific IgA capturing antibody to the plastic surface of the 96-well microtiter plate (pre-coating plate). The process further comprises a second phase that comprises two stages. The first stage comprises the binding of IgA molecules present in a tested samples to the pre-adsorbed IgA capturing antibody. The second stage may comprise a two-step detection of human Gd-IgA1 molecules captured on the microtiter plate. The first step comprises binding the captured human galactose-deficient IgA1 (Gd-IgA1) of the sample with biotin-labeled HPA lectin. In the second step, biotin is detected by a peroxidase-labeled avidin through a colorimetric product generated through a reaction of the peroxidase substrate, 3,3′,5,5′-tetramethylbenzidine (TMB), measured at 450 nm. The absorbance at 450 nm is proportional to the amount of total Gd-IgA1 antibodies present in human serum based on included calibrators and validation studies of the assay. The process is capable of being performed on multiple plates in parallel, but a separate GalD Plate Map and Performance File should be used for each assay plate.

In some embodiments, the entire kit may be stored at temperatures of −20° C. In some embodiments, the 20× first washing buffer, reagent buffer/second wash buffer additive, and STOP solution should be stored at room temperature (RT: 20-25° C. (68-77°) F)), the second reagent and third reagent should be stored at 4° C. and the Plate, reagent buffer, first reagent, standard, and quality controls 1-3 should be stored at −20° C.

In some embodiments, a kit for detecting the presence of one or more galactose-deficient IgA1 proteoforms in a biological sample comprises: a calibration standard comprising one or more of enzymatically modified IgA1 antibodies, recombinantly produced Galactose deficient-IgA1 (Gd-IgA1) that is enzymatically modified to present Gd-IgA1, recombinantly produced Gd-IgA1, synthetically produced IgA1, IgA1 heavy chain, or IgA1 hinge region with extended domains on N- or C-terminal portions. In some embodiments, the calibration standard of the kit has been benchmarked relative to a well-characterized Gd-IgA1 reference material. In some embodiments, the well-characterized Gd-IgA1 reference material has a measurable relative abundance of terminal-GalNAc O-glycans of 20-80% compared to non-terminal-GalNAc O-glycans. In some embodiments, the kit may further comprise; a capture reagent, wherein the capture reagent is an IgA-specific capture reagent; a detection reagent, wherein the detection reagent is a lectin having standardized binding activity to more than one type of galactose-deficient IgA1 proteoforms. In some embodiments, the kit of may further comprise: an enzyme conjugate, wherein the enzyme conjugate is a conjugation that creates a measurable colorimetric signal. In some embodiments, the kit of may further comprise: a substrate, wherein the substrate is a chromogenic, luminescence, or fluorescence buffer. In some embodiments, the kit of may further comprise: a first wash buffer, a second wash buffer, a stop solution, a reagent buffer and additive.

### Methods of Using an Assay Kit for the Quantification of GalD IgA1

In some embodiments, a process of quantifying gal-deficient IgA1 in patients via an N-acetylgalactosamine (GalNAc)-specific lectin-based Gd-IgA1 assay (GalD Assay) is provided (i.e. administering the GalD Assay). The GalD Assay is an indirect sandwich ELISA (Enzyme-linked Immunosorbent Assay) assay that quantitatively measures levels of GdlgA1 (Galactose-deficient lgA 1) in human serum samples. In some embodiments, the method of administering the assay may be incorporated into a decision tree or diagnostic tree for one or more kidney diseases, wherein the method of administering the assay is capable of being incorporated into a decision tree or diagnostic tree for one or more kidney diseases. The embodiments of the assay, the embodiments of the method of administering the assay and/or the embodiments of the method of quantification of Gal-deficient IgA1 using the assay may be used in a decision tree to guide kidney transplantation and/or screen kidney donors and transplant organs. The assay process generally comprises a two-step detection reaction. In the first step, a biotin-labeled lectin selectively binds to aberrantly glycosylated IgA1. In the second step, biotin is detected by peroxidase-labeled avidin in a colorimetric reaction of tetra-methylbenzidine (TMB) and measured at 450 nm.

The assay kit and related processes may comprise one or more of the following: a precoated plate, a first wash buffer solution (W1), a second wash buffer solution (W2), a reagent buffer solution (RB), a reagent buffer/second wash buffer additive solution (RB/W2 Add.), a first reagent solution (R1), a second reagent solution (R2), a third reagent solution (R3), a stop solution (STOP), a standard solution (STD), a first quality control solution (QC1), a second quality control solution (QC2), a third quality control solution (QC3).

In some embodiments, a first wash buffer solution (W1) is a 20× phosphate buffer. In some embodiments, a reagent buffer solution (RB) is a 10% Bovine serum albumin. In some embodiments, a stop solution (STOP) is a tetramethylbenzidine (TMB) liquid. In some embodiments, a reagent buffer/second wash buffer additive solution (RB/W2 Add.) is 25% Tween 20. In some embodiments, a second reagent solution (R2) is ExtrAvidin peroxidase. In some embodiments, the Plate is a 96-well plate.

The assay kit and related processes may further comprise one or more of the following: microplate reader capable of measuring absorbance at 450 nm, microcentrifuge, pipettes, pipette tips, 1 L graduated cylinders, 1 L containers/bottles, 15 ml containers/tubes, and biotech grade water (type 1), a microplate washer.

The process of kit preparation may comprise the following steps: acclimating the plate to room temperature without removing the seal; removing the seal before initial washing; preparing the W1, W2, and complete RB buffer solutions. The prepared W1, W2 and complete RB buffer may be kept at room temperature until the end of the assay process. The preparation of W1 comprises mixing 570 ml of water and 30 mL of 20×W1. The preparation of W2 comprises mixing 570 ml of water, 30 mL of 20×W1 and 1.2 ml of RB/W2 Add. The preparation of complete RB buffer comprises mixing 12 mL of RB solution, 102 mL of water, 6 ml of 20×W1 and 220 μl of RB/W2 Add. All other reagents should be prepared at the time of their use. The preparation of R1 comprises reconstitution of an R1 pellet in water (for example, 100 μl) before adding complete RB buffer (for example, 12 mL). The preparation of R2 comprises spinning 10 μl of R2 solution briefly before mixing it with 12 mL of complete RB buffer. In some embodiments, the spinning occurs until the solution is collected. The preparation of R3 comprises mixing 6 ml of R3 solution with 6 ml of water. The preparation of STD 0 comprises reconstitution of a pellet of STD 0 in 250 μl of water. The concentration of STD 0 is 4000 ng/ml. The preparation of QC 1-3 comprises reconstitution of a QC pellet in 250 μl of water.

The process of preparation of a Standard Curve may comprise the following steps: Prepare 7 tubes, labeled from STD 1 to STD 7, for 2× serial dilution by adding 250 μl of complete RB to each tube; take 250 μl of STD O and add it to the STD 1 tube; mix by pipetting it up and down at least 5 limes (avoid bubbles), and repeat transfer of 250 μl and mixing for each sequentially to STD 7. This gives a set of 7 standards (250 μl each) to load on the plate. An example of OD values for each concentration (OD values may vary) log of concentrations, and four parameters logistic standard curve are shown in FIG. 3.

The process of sample preparation may comprise the following steps: Performing all serum sample dilutions at room temperature (20-25° C. (68-77°) F); Mixing serum samples thoroughly; taking an aliquot of the mixed samples for dilution; diluting serum samples to 2000× dilution with complete RB. For example, make an initial 100× dilution by taking 5 μL of serum to 495 μL of complete RB, then, take 20 μL of 100× diluted samples and add it to 380 μL of complete RB. Any samples with an OD reading above the highest calibrator (STD 1) should be reassayed at a greater dilution. Samples may be run either in duplicates or triplicates and averaged.

The process of administering the assay may comprise the following steps: acclimating the plate to room temperature (20-25° C.; 68-77° F.) for about 15 minutes. In some embodiments, the plate is covered while it acclimates; Washing the plate wells (Wash #1). In some embodiments, plate wells are washed one, two, three, four, five or more times with as washing solution such as 300 μL of W1; (3) reloading the plate with 100 μL of complete RB (whole plate); adding 100 μL of standards (STD 1-7), 100 μL of QCs, 100 μL of complete RB (blank/BNK), or 100 μL of samples (at 2000× dilution) to the appropriate wells as shown in Table 6 (the total volume of each preloaded well after the appropriate solutions are added is 200 μL); incubating the plate for 1 hour at room temperature; washing the plate (Wash #2). In some embodiments, plate wells are washed one, two, three, four, five or more times with as washing solution such as 300 μL of W2; adding 100 μL of R1 to each well and incubating for 1 hour at room temperature (Incubation #2); washing the plate (Wash #3). In some embodiments, plate wells are washed one, two, three, four, five or more times with as washing solution such as 300 μL of W2. Add 100 μL of R2 to each well and incubate for 1 h al RT (Incubation #3); washing the plate wells (Wash #4). In some embodiments, plate wells are washed one, two, three, four, five or more times with as washing solution such as 300 μL of W1; Removing excess liquid from the wells; adding 100 μL of R3 to each well and incubate for 30 min at room temperature in the dark (Incubation #4); adding 100 μL of STOP solution to each well; measuring optical density at 450 nm. In some embodiments,

| | 1 | 2 | 3 | 4 | 5 | 6 | 7 | 8 | 9 | 10 | 11 | 12 |
|---|---|---|---|---|---|---|---|---|---|---|---|---|
| A | STD 1 | STD 1 | SPL 1 | SPL 1 | SPL 5 | SPL 5 | SPL 13 | SPL 13 | SPL 21 | SPL 21 | SPL 29 | SPL 29 |
| | 2000 | 2000 | | | | | | | | | | |
| B | STD 2 | STD 2 | SPL 2 | SPL 2 | SPL 6 | SPL 6 | SPL 14 | SPL 14 | SPL 22 | SPL 22 | SPL 30 | SPL 30 |
| | 1000 | 1000 | | | | | | | | | | |
| C | STD 3 | STD 3 | SPL 3 | SPL 3 | SPL 7 | SPL 7 | SPL 15 | SPL 15 | SPL 23 | SPL 23 | SPL 31 | SPL 31 |
| | 500 | 500S | | | | | | | | | | |
| D | STD 4 | STD 4 | SPL 4 | SPL 4 | SPL 8 | SPL 8 | SPL 16 | SPL 16 | SPL 24 | SPL 24 | SPL 32 | SPL 32 |
| | 250 | 250 | | | | | | | | | | |
| E | STD 5 | STD 5 | QC 1 | QC 1 | SPL 9 | SPL 9 | SPL 17 | SPL 17 | SPL 25 | SPL 25 | SPL 33 | SPL 33 |
| | 125 | 125 | | | | | | | | | | |
| F | STD 6 | STD 6 | QC 2 | QC 2 | SPL 10 | SPL 10 | SPL 18 | SPL 18 | SPL 26 | SPL 28 | SPL 34 | SPL 34 |
| | 62.5 | 62.5 | | | | | | | | | | |
| G | STD 7 | STD 7 | QC 3 | QC 3 | SPL 11 | SPL 11 | SPL 19 | SPL 19 | SPL 27 | SPL 27 | SPL 35 | SPL 35 |
| | 31.25 | 31.25 | | | | | | | | | | |
| H | BLK | BLK | BLK | BLK | SPL 12 | SPL 12 | SPL 20 | SPL 20 | SPL 28 | SPL 28 | SPL 36 | SPL 36 |

it is optimal to measure optical density within 30 minutes of completion of the previous step.

In Table 6, STD (standards 1-7) contains STD samples prepared; QC (quality controls 1-3) contains QC samples prepared; BLN (blanks): contains only complete RB (200 μl); SPL (samples 1-36) contains samples prepared.

QCs should be run in duplicates. QCs should be run on each plate. The concentration of QC (read directly from the standard curve) should be within the following range: QC1=653-884 ng/ml; QC2=426-578 ng/mL; QC3=245-332 ng/mL.

The process of calculating results of a completed assay may comprise one or more of the following steps: Fit the concentrations of standards and their optical density values using a four parameter logistic curve, ensuring the standard curve maintains an R2 value of 0.98, which is considered a good fit; Read the concentration of samples by applying their ODs to the standard curve; Multiply the concentration of samples by their dilution (2000×).

In some embodiments the assay has sensitivity of 125 ng/mL. In some embodiments the assay has measurement range of 125-2,000 ng/mL. In some embodiments the assay has sensitivity of 250-4000 ng/mL.

For the purposes of promoting an understanding of the principles of the invention, reference has been made to the preferred embodiments illustrated in the drawings, and specific language has been used to describe these embodiments. However, this specific language intends no limitation of the scope of the invention, and the invention should be construed to encompass all embodiments that would normally occur to one of ordinary skill in the art. The particular implementations shown and described herein are illustrative examples of the invention and are not intended to otherwise limit the scope of the invention in any way. For the sake of brevity, conventional aspects of the system (and components of the individual operating components of the system) may not be described in detail. Furthermore, the connecting lines, or connectors shown in the various figures presented, are intended to represent exemplary functional relationships and/or physical or logical couplings between the various elements. It should be noted that many alternative or additional functional relationships, physical connections, or logical connections may be present in a practical device. Moreover, no item or component is essential to the practice of the invention unless the element is specifically described as “essential” or “critical”. Numerous modifications and adaptations will be readily apparent to those skilled in this art without departing from the spirit and scope of the present invention.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. A calibration standard for use in an assay comprising:
One or more of enzymatically modified IgA1 antibodies, recombinantly produced Galactose deficient-IgA1 (Gd-IgA1) that is enzymatically modified to present Gd-IgA1, recombinantly produced Gd-IgA1, synthetically produced IgA1, IgA1 heavy chain, or IgA1 hinge region with extended domains on N- or C-terminal portions.

33. The calibration standard of claim 32, wherein the calibration standard is benchmarked relative to a well-characterized Gd-IgA1 reference material with known IgA1 concentration.

34. The calibration standard of claim 33, wherein the well-characterized Gd-IgA1 reference material has a measurable relative abundance of terminal-GalNAc O-glycans of 20-80% compared to non-terminal-GalNAc O-glycans.

35. The calibration standard of claim 34, wherein the standard is enzymatically treated serum with up to 50% recombinantly produced Gd-IgA1.

36. The calibration standard of claim 35, wherein the standard is enzymatically treated with neuraminidase.

37. The calibration standard of claim 32, further comprising naturally occurring Galactose deficient-IgA1 antibodies (Gd-IgA1).

38. The calibration standard of claim 32, wherein the calibration standard exhibits parallelism with both the well-characterized Gd-IgA1 reference material as well as non-modified human serum samples when combined with a standardized HPA lectin.

39. The calibration standard of claim 32, wherein the calibration standard is stable over time.

40. The calibration standard of claim 32, wherein the calibration standard is configured to be used in assays for the measurement of one or more galactose-deficient IgA1 proteoforms.

41. A method of detecting gal-deficient IgA1 in a biological sample, comprising;
a. Isolating the IgA1 antibodies present in the biological sample by contacting the biological sample with an IgA1-specific capture reagent to create IgA1 complexes;
b. Contacting the IgA1 complexes with a detection reagent to form sandwich complexes, wherein the detection reagent is configured to bind to more than one type of galactose-deficient IgA1 proteoforms;

42. The method of claim 41, wherein the detection reagent is a standardized lectin that has been standardized using a calibration standard comprising one or more of a naturally occurring Gd-IgA, enzymatically modified IgA1, recombinantly produced IgA1 that is enzymatically modified to present Gd-IgA1, recombinantly produced Gd-IgA1, synthetically produced IgA1, IgA1 heavy chain, or IgA1 hinge region with extended domains on N- or C-terminal portions.

43. The method of claim 41, further comprising the step of:
c. measuring a signal resulting from the presence of the sandwich complexes.

44. The method of claim 43, wherein the measurable signal is a colorimetric reaction produced by contacting the sandwich complexes with an enzyme conjugate to form a conjugated complex and then contacting the conjugated complex with a substrate.

45. A method of standardizing a test batch of a lectin to a reference batch of lectin for use as a reagent comprising:
a. Providing an amount of a reference batch of lectin with a known binding activity;
b. Providing an amount of the test batch of lectin;
c. Providing a calibration standard to calibrate the activity of a lectin molecule from any source for the detection of its natural glycan target in a biological sample.
d. Preparing at least a portion of the test batch of lectin into one or more aliquots of known concentrations of the test batch lectin;
e. Performing a test to assess the binding activity of the one or more aliquots of the test batch lectin and the reference batch lectin to the calibration standard.
f. selecting the one or more known concentrations of the test batch lectin with binding activity most similar to the reference batch of lectin for use as a reagent,

46. The method of claim 45, wherein the similarity of the one or more known concentrations of test batch lectin and reference lectin are determined by plotting a measured signal correlating to binding activity for the test batch and reference batch and comparing the plots.

47. The method of claim 46, wherein selecting the one or more known concentrations of the test batch lectin with binding activity most similar to the reference batch of lectin is based on an acceptance criteria, wherein the acceptance criteria comprises the test batch lectin having a plotted measured signal with an analytical recovery within 15% of the plotted measured signal of the reference batch, wherein analytical recovery is defined as the actual value divided by the measurement multiplied by 100%.

48. The method described in claim 47 wherein if the one or more known concentrations of test batch lectin do not have a plotted measured signal that satisfies the acceptance criteria, the method comprises the additional step of:
g. selecting the known concentration of test batch lectin that has a plotted measured signal that is the closest to satisfying the acceptance criteria and creating a subsequent set of further known concentrations from said known concentration wherein the subsequent range of further known concentrations is one of:
a range having the best-fit known concentration as a midpoint,
a range having the best-fit known concentration as a midpoint, wherein the range is narrower than the range of a previous test,
a range wherein an endpoint of the range is the best-fit known concentration, and;
Performing steps a through f again using the reference batch of lectin and further known concentrations of the test batch of lectin,
h. Repeating step a-g, until a known concentration or further known concentration of the test batch of lectin produces a plotted measured signal that satisfies the acceptance criteria.

49. The method of claim 48, wherein the measured signal is the optical density of a produced colorimetric reaction.

50. The method of claim 45, wherein the biological sample comprises one or more of serum, urine, other biological fluid, a cell sample, a cell secretion, cell isolate, or other cell preparations.

51. The method of claim 45, wherein the calibration standard is a well-characterized standard of the natural glycan presented in a biologically relevant molecule.

52. The method of claim 45, wherein the calibration standard comprises one or more of a naturally occurring Gd-IgA, enzymatically modified IgA1 (that allows presentation of Gd-IgA1), recombinantly produced IgA1 that is enzymatically modified to present Gd-IgA1, or recombinantly produced Gd-IgA1.

53. The method described in claim 45, wherein the known concentrations of test batch of lectin, comprise an amount of 1% to 500% of the test batch of lectin.

54. A method of standardizing a test batch of a biomarker, peptide or protein, to a reference batch of biomarker, peptide or protein, for use as a reagent comprising:
a. Providing an amount of a reference batch of biomarker, peptide or protein, with a known binding activity to a target analyte;
b. Providing an amount of the test batch of biomarker, peptide or protein;
c. Providing a calibration standard has been benchmarked to a well-characterized reference material comprising at least the target analyte;
d. Preparing at least a portion of the test batch into one or more aliquots of known concentrations;
e. Performing a test to assess the binding activity of the one or more aliquots of the test batch and the reference batch to the calibration standard.
f. selecting the one or more known concentrations of the test batch with binding activity most similar to the reference batch for use as a reagent,

55. The method of claim 54, wherein the similarity of the one or more known concentrations of test batch and reference are determined by plotting a measured signal correlating to binding activity for the test batch and reference batch and comparing the plots for best-fit.

56. The method of claim 55, wherein selecting the one or more known concentrations of the test batch with binding activity most similar to the reference batch is based on an acceptance criteria, wherein the acceptance criteria comprises the test batch having a plotted measured signal with an analytical recovery within 15% of the plotted measured signal of the reference batch, wherein analytical recovery is defined as the actual value divided by the measurement multiplied by 100%.

57. The method described in claim 56, wherein if the one or more known concentrations of test batch do not have a plotted measured signal that satisfies the acceptance criteria, the method comprises the additional step of:
g. selecting the known concentration of test batch that has a plotted measured signal that is the closest to satisfying the acceptance criteria and creating a subsequent set of further known concentrations from said known concentration wherein the range of the further known concentrations is one of:
a range having the best-fit known concentration as a midpoint,
a range having the best-fit known concentration as a midpoint, wherein the range is narrower than the range of a previous test,
a range wherein an endpoint of the range is the best-fit known concentration, and;
Performing steps a through f again using the reference batch and further known concentrations of the test batch.
h. Repeating step a-g, until a known concentration or further known concentration of the test batch produces a plotted measured signal that satisfies the acceptance criteria.

58. The method of claim 56, wherein the measured signal is the optical density of a produced colorimetric reaction.

59. The method of claim 55 wherein the biological sample comprises one or more of serum, urine, other biological fluid, a cell sample, a cell secretion, cell isolate, or other cell preparations.

60. The method of claim 55, wherein the calibration standard comprises one or more of enzymatically modified IgA1 antibodies, recombinantly produced Galactose deficient-IgA1 (Gd-IgA1) that is enzymatically modified to present Gd-IgA1, recombinantly produced Gd-IgA1, synthetically produced IgA1, IgA1 heavy chain, or IgA1 hinge region with extended domains on N- or C-terminal portions.

61. The method described in claim 55, wherein the known concentrations of the test batch comprises an amount of 1% to 500% of the reference batch.
